(19) United States
(12) Patent Application Publication
(10) Pub. No.: US 2003/0228677 A1
(43) Pub. Date: Dec. 11, 2003

(54) AMID protein, nucleic acid molecules, and uses thereof

(76) Inventors: Hong-Bing Shu, Denver, CO (US); Liang-Guo Xu, Denver, CO (US); Min Wu, Beijing (CN)

Correspondence Address: SHERIDAN ROSS PC, 1560 BROADWAY, SUITE 1200, DENVER, CO, 80202

(21) Appl. No.: 10/434,418

(22) Filed: May 7, 2003

Related U.S. Application Data
(60) Provisional application No. 60/378,752, filed on May 7, 2002.

(51) Int. Cl. C12N 9/64; C07H 21/04; C12P 21/02; C12N 5/06

(52) U.S. Cl. 435/226; 435/69.1; 435/320.1; 435/325; 536/23.2

(57) ABSTRACT

Described here is the cloning and characterization of a novel AIF homologous molecule designated AMID, which is co-localized with mitochondria, and which induces cell death with characteristic apoptotic morphology, independent of caspase activation and p53. Bcl2. Disclosed herein are AMID proteins, fragments and homologues, as well as nucleic acid molecules encoding such proteins, and methods of making and using the same.

### CROSS-REFERENCE TO RELATED APPLICATIONS

This application claims the benefit of priority from U.S. Provisional Application Serial No. 60/378,752, filed May 7, 2002, entitled, “AMID Protein, Nucleic Acid Molecules, and Uses Thereof.” The entire disclosure of U.S. Provisional Application Serial No. 60/378,752 is incorporated by reference herein.

### FIELD OF THE INVENTION

This invention generally relates to a novel protein involved in caspase-independent apoptosis, called AMID, and to nucleic acid molecules encoding AMID, and to methods of making and using AMID.

### BACKGROUND OF THE INVENTION

Apoptosis, or programmed cell death, is one of the fundamental processes in all metazoans. It is widely believed that the mitochondrion is an integrator of the cell death machinery and caspases are the central executioners of apoptosis (Green (1999) Science 281, 1309-1312; Hengartner (2000) Nature 407, 770-776; Strasser (2000) Annu. Rev. Biochem. 69, 217-245; Daugas (2000) FEBS Lett. 476, 118-123; Wang (2001) Genes Dev. 22, 2922-2933; Hunot (2001) Science 292, 865-866). Upon stimulation by various death signals, the outer membrane of the mitochondrion is permeabilized, resulting in the release to the cytosol of molecules including cytochrome c and Smac/DIABLO) (Green, supra; Hengartner, supra; Strasser, supra; Daugas, supra; Wang, supra; Hunot, supra; Liu (1996) Cell 86, 147-157; Du (2000) Cell 102, 33-42; Verhagen (2000) Cell 102, 43-53). Once released into the cytosol, cytochrome c binds to Apaf-1 and triggers the oligomerization of Apaf-1, which in turn recruits pro-caspase-9 to the “apoptosome” complex (Zou (1999) J. Biol Chem. 274, 11549-11556; Rodriguez (1999) Genes and Deve. 13, 3179-3184). Recruitment of pro-caspase-9 into the “apoptosome” causes its auto-activation and further activation of downstream executioner caspases such as caspase-3 (Zou, supra; Rodriguez, supra). The activated executioner caspases cleave various cellular substrates, leading to characteristic morphological changes in apoptosis such as chromatin condensation, nucleosomal DNA fragmentation, and formation of apoptotic bodies (Green, supra; Hengartner, supra; Strasser, supra; Daugas, supra; Wang, supra; Hunot, supra).

Death signal-induced release of Smac from mitochondrion provides an alternative mechanism of cell death regulation. It has been proposed that cytosolic Smac released from mitochondrion interacts with inhibitors of apoptosis protein (IAPs), competes IAP's interaction with activated caspases, and therefore promotes apoptosis (Du, supra; Verhagen, supra).

Although cytochrome c and caspases are critically involved in the classic apoptotic pathways, various studies indicate that cytochrome c, Apaf-1, or caspase deficient cells can still undergo apoptosis (Li (2000) Cell 101, 389-399; Yoshida (1998) Cell 94, 739-750; Cecconi (1998) Cell 94, 727-737; Hakem (1998) Cell 94, 339-352; Kuida (1996) Nature 384, 368-372; Kuida (1998) Cell 94, 325-337). In addition, numerous studies have demonstrated that apoptosis induced by some stimuli can not be inhibited by the pan-caspase inhibitor z-VAD.fmk (Amarante-Mendes (1998) Cell Death Differ. 5, 298-306; Haraguchi (2000) J. Exp. Med. 191, 1709-1720; Bidere (2001) Apoptosis 6, 371-375), suggesting that caspase-independent apoptotic pathways exist.

In a search for molecules that cause caspase-independent apoptosis, Susin et al. identified apoptosis-inducing factor (AIF) (Susin (1999) Nature 397, 441-446). AIF is a 57 kDa flavoprotein with an ˜100aa mitochondrial localization sequence (MLS) at its N-terminus. Like cytochrome c, AIF is normally present in the intermembrane space of mitochondria. Upon stimulation by death signals, AIF translocates from the mitochondria to the nucleus and causes chromosome condensation and large-scale DNA fragmentation (Susin, supra; Daugas (2000) FASEB J. 14, 729-739). These effects are independent of caspases and the oxidoreductase activity of AIF (Susin, supra; Daugas, supra; Joza, N., Susin (2001) Nature 410, 549-554; Loeffler (2001) FASEB J. 15, 758-767; Miramar (2001) J Biol. Chem. 276, 16391-16398). Gene knock-out studies further suggest that AIF is required for apoptosis of embryonic stem cells induced by serum deprivation, and is essential for apoptosis during cavitation of embryonic bodies (Joza, supra). Moreover, these experiments also indicate that AIF-induced apoptosis can be genetically uncoupled from those induced by Apaf-1 and caspase-9 (Joza, supra).

Apoptosis is intricately involved in various physiological and pathological processes, including embryonic development, immunological regulation, and tumorogenesis. The ability to regulate apoptosis in cells or to identify proteins that play a role in apoptosis, particularly in certain cell types, is therefore important. The identification of new genes and proteins that control apoptotic processes in a cell is valuable for the design of tools and for the identification of targets for the regulation of apoptosis for diagnostic and therapeutic purposes.

### SUMMARY OF THE INVENTION

One embodiment of the invention relates to an isolated protein comprising an amino acid sequence selected from the group consisting of: (a) an amino acid sequence comprising SEQ ID NO:2; (b) a biologically active fragment of SEQ ID NO:2; and (c) an amino acid sequence that is at least about 50% identical to SEQ ID NO:2, wherein the amino acid sequence has a biological activity of SEQ ID NO:2. In other aspects, the protein comprises an amino acid sequence that is at least about 70% identical or at least about 90% identical to SEQ ID NO:2, wherein the protein has a biological activity of SEQ ID NO:2. Fragments of SEQ ID NO:2 include, but are not limited to, a fragment of SEQ ID NO:2 comprises an amino acid sequence spanning from a starting position of between amino acid 2 and amino acid 185 of SEQ ID NO:2, to an ending position of amino acid 373 of SEQ ID NO:2, positions 37-373 of SEQ ID NO:2, positions 41-373 of SEQ ID NO:2, positions 77-373 of SEQ ID NO:2, and positions 186-373 of SEQ ID NO:2. In one aspect, the protein comprises an amino acid sequence that is at least 50% identical and less than 100% identical, less than 95% identical, or less than 90% identical to SEQ ID NO:2, wherein the protein has a biological activity of SEQ ID NO:2. In one aspect, the biological activity is apoptosis-inducing activity.

Another embodiment relates to an isolated fusion protein comprising a first amino acid sequence as described above, and a second amino acid sequence that is heterologous to the first amino acid sequence, wherein the first and second are linked to form a fusion protein.

Yet another embodiment of the invention relates to an isolated antibody or antigen binding fragment thereof that selectively binds to SEQ ID NO:2. Also included is a diagnostic kit comprising at least one of such antibodies or antigen binding fragments.

Another embodiment of the present invention relates to a composition comprising at least about 1 μg of an isolated protein selected from the group consisting of: (a) an amino acid sequence comprising SEQ ID NO:2; (b) a biologically active fragment of SEQ ID NO:2; and (c) an amino acid sequence that is at least about 50% identical to SEQ ID NO:2, and has a biological activity of SEQ ID NO:2.

Another embodiment of the present invention relates to an isolated nucleic acid molecule consisting essentially of a nucleic acid sequence selected from the group consisting of: (a) a nucleic acid sequence encoding SEQ ID NO:2; (b) a nucleic acid sequence encoding a biologically active fragment of SEQ ID NO:2; (c) a nucleic acid sequence encoding an amino acid sequence that is at least about 50% identical to SEQ ID NO:2 and has a biological activity of SEQ ID NO:2; and (d) a nucleic acid sequence that is fully complementary to any of the nucleic acid sequences of (a)-(c). In one aspect, the nucleic acid sequence encodes an amino acid sequence that is at least about 70% identical or at least about 90% identical to SEQ ID NO:2 and has a biological activity of SEQ ID NO:2. In one aspect, the fragment of SEQ ID NO:2 includes, but is not limited to, a fragment of SEQ ID NO:2 spanning from a starting position of between amino acid 2 to amino acid 185 of SEQ ID NO:2, to an ending position of amino acid 373 of SEQ ID NO:2, a fragment of SEQ ID NO:2 spanning positions 77-373 of SEQ ID NO:2, or a fragment of SEQ ID NO:2 spanning positions 186-373 of SEQ ID NO:2. In another embodiment, the nucleic acid sequence encodes a protein having an amino acid sequence that is at least 50% identical and less than 100% identical to SEQ ID NO:2, wherein the protein has a biological activity of SEQ ID NO:2. In one aspect, the nucleic acid sequence encodes SEQ ID NO:2. In another aspect, the nucleic acid molecule consists essentially of SEQ ID NO:5.

Also included in the invention are a recombinant nucleic acid molecule comprising any of the above-identified isolated nucleic acid molecules of the invention and a heterologous vector sequence. In one embodiment, the nucleic acid molecule is operatively linked to a transcription control sequence. The present invention also includes an isolated host cell transfected with the recombinant nucleic acid molecule described herein.

Another embodiment of the invention relates to an oligonucleotide consisting essentially of at least 22 consecutive nucleotides of SEQ ID NO: 1 or SEQ ID NO:5, or the complement thereof. Also included is a diagnostic kit comprising at least one such oligonucleotide.

Yet another embodiment of the invention relates to a method for detecting the presence of a cancer in a patient, comprising the steps of: (a) obtaining a biological sample comprising cells from a patient; (b) detecting in the cells the expression of a protein comprising SEQ ID NO:2 or a nucleic acid molecule comprising SEQ ID NO:5; and (c) comparing the level of expression of the protein or nucleic acid molecule detected in (b) to a control expression level as an indicator of the presence of a cancer in the patient, wherein a decrease in the expression of the protein or the nucleic acid molecule as compared to the control is an indicator of a positive diagnosis of cancer in the patient. In one aspect, the step of detecting comprises contacting the biological sample with a binding agent that binds to a protein comprising SEQ ID NO:2, and detecting in the sample an amount of protein that binds to the binding agent. The binding agent can include, but is not limited to: an antibody, an antigen binding fragment, and a peptide that selectively binds to SEQ ID NO:2. In one aspect, the step of contacting comprises contacting the cells in the biological sample intracellularly with the binding agent. In another aspect, the step of detecting comprises contacting nucleic acids in the biological sample with an oligonucleotide consisting essentially of at least 22 consecutive nucleotides of SEQ ID NO:5, or the complement thereof, and detecting in the sample an amount of nucleic acids that hybridize to the oligonucleotide under highly stringent conditions. This step of contacting can include contacting the cells in the biological sample intracellularly with the oligonucleotide. In one aspect, the step of detecting comprises detecting expression of an RNA sequence comprising SEQ ID NO:5 or a fragment thereof in the biological sample as compared to expression of the RNA sequence in the control. In another aspect, the step of detecting comprises detecting a nucleic acid sequence in a sample comprising a cDNA product of RNA comprising SEQ ID NO:5 or a fragment thereof as compared to a cDNA product of RNA in the control. In another aspect, the step of detecting comprises detecting a nucleic acid sequence in a sample comprising amplified nucleic acid products of RNA comprising SEQ ID NO:5 or a fragment thereof in the sample as compared to amplified nucleic acid products of RNA from the control.

Another embodiment of the present invention relates to a method for inducing apoptosis in a cell, comprising contacting a cell intracellularly with a compound selected from the group consisting of: (a) an isolated protein comprising an amino acid sequence selected from the group consisting of: (i) an amino acid sequence comprising SEQ ID NO:2; (ii) a biologically active fragment of SEQ ID NO:2; and (iii) an amino acid sequence that is at least about 50% identical to SEQ ID NO:2, wherein the amino acid sequence has a biological activity of SEQ ID NO:2; or (b) an isolated nucleic acid molecule comprising a nucleic acid sequence encoding a protein of (a), the nucleic acid sequence being operatively linked to a transcription control sequence; or (c) a product of drug discovery that has the biological activity of a protein comprising SEQ ID NO:2.

Yet another embodiment of the present invention relates to a method for the treatment of cancer, comprising contacting tumor cells in a patient with a compound, under conditions and for a time sufficient to permit the stimulation of the apoptotic process, the compound being selected from the group consisting of: (a) an isolated protein comprising an amino acid sequence selected from the group consisting of: (i) an amino acid sequence comprising SEQ ID NO:2; (ii) a biologically active fragment of SEQ ID NO:2; and (iii) an amino acid sequence that is at least about 50% identical to SEQ ID NO:2 and having a biological activity of SEQ ID NO:2; or (b) an isolated nucleic acid molecule comprising a nucleic acid sequence encoding a protein of (a), the nucleic acid sequence being operatively linked to a transcription control sequence; or (c) isolated antigen-presenting cells transfected with and expressing the isolated nucleic acid molecule of (b); or (d) a product of drug discovery that has the biological activity of a protein comprising SEQ ID NO:2. In one aspect, the treatment is combined with chemotherapy, tumor excision, radiation therapy or other cancer therapy.

Another embodiment of the present invention relates to a method for reducing apoptosis in a cell, comprising contacting a cell intracellularly with a compound selected from the group consisting of: (a) an antibody or antigen binding fragment thereof that selectively binds to and inhibits the activity of a protein comprising SEQ ID NO:2; (b) an isolated protein that is at least about 50% identical and less than about 100% identical to SEQ ID NO:2, wherein the protein is an antagonist of a protein comprising SEQ ID NO:2; (c) a fragment of SEQ ID NO:2 that has reduced apoptosis-inducing biological activity as compared to SEQ ID NO:2; (d) an isolated nucleic acid sequence comprising at least 22 consecutive nucleotides of SEQ ID NO: 1 or SEQ ID NO:5 and that hybridizes under highly stringent conditions to a gene encoding SEQ ID NO:2 and inhibits the expression of a protein comprising SEQ ID NO:2; or (e) a product of drug discovery that inhibits the activity of a protein comprising SEQ ID NO:2 or the expression of a gene encoding a protein comprising SEQ ID NO:2.

Another embodiment of the present invention relates to a method to identify a regulator of apoptosis, comprising: (a) contacting an apoptosis-inducing protein or nucleic acid molecule encoding the apoptosis-inducing protein with a putative regulatory compound, wherein the apoptosis-inducing protein comprises an amino acid sequence selected from the group consisting of: (i) an amino acid sequence comprising SEQ ID NO:2; (ii) a biologically active fragment of SEQ ID NO:2; and (iii) an amino acid sequence that is at least about 50% identical to SEQ ID NO:2 and has a biological activity of SEQ ID NO:2; and (b) detecting whether the putative regulatory compound increases or decreases expression or activity of the protein or the nucleic acid molecule as compared to prior to contact with the compound. Compounds that increase or decrease the expression or activity of the protein or nucleic acid molecule, as compared to in the absence of the compound, indicates that the putative regulatory compound is a regulator of apoptosis. In one aspect, the apoptosis-inducing protein is expressed by a cell, and wherein the putative regulatory compound is contacted with the protein or nucleic acid molecule intracellularly. In another aspect, the step of detecting comprises detecting binding or association of the putative regulatory compound with the protein or nucleic acid molecule. In yet another aspect, the step of detecting comprises detecting the ability of the putative regulatory compound to regulate apoptosis in a cell that expresses the protein or nucleic acid molecule.

### BRIEF DESCRIPTION OF THE DRAWINGS

FIG. 1 is an alignment of amino acid sequences of human AMID (SEQ ID NO:2), human AIF (SEQ ID NO:3) and E. Coli NADH-oxidoreductases (SEQ ID NO:4). The three possible starting methionines are underlined.

FIG. 2A is a schematic presentation of AMID deletion mutants.

FIG. 2B is a bar graph showing the effects of AMID deletion mutants on apoptosis.

FIG. 3A is a bar graph showing the effects of crmA and the pan-caspase inhibitor VAD-fmk on AMID- and FADD-induced apoptosis.

FIG. 3B is a bar graph showing that AMID-induced apoptosis is independent of p53.

FIG. 4 is a bar graph showing that overexpression of AIF is insufficient to induce apoptosis.

### DETAILED DESCRIPTION OF THE INVENTION

The present invention relates generally to proteins that play a role in the regulation of apoptosis, designated AMID (AIF-homologous Mitochondrion-associated Inducer of Death), and to homologues of AMID, fusion proteins comprising AMID, and nucleic acid molecules encoding such proteins and homologues. The invention also relates to a diagnostic assay and method of therapy for cancer, including, but not limited to, colon, kidney and stomach cancer. The proteins and nucleic acids of the invention may be used in pharmaceutical compositions for prevention and treatment of tumors, for the diagnosis and monitoring of such cancers, as well as in other methods of inducing or reducing apoptosis in a cell. The proteins and nucleic acids of the invention may also be used in methods for identifying compounds that inhibit or induce apoptosis via action on AMID.

The mitochondrion plays a central role in apoptosis induced by various stimuli. Upon stimulation by death signals, the mitochondrion releases to the cytosol molecules including cytochrome c, Smac/DIABLO, and AIF, among others. Once released into the cytosol, cytochrome c and Smac trigger well-defined caspase-dependent apoptotic pathways. In contrast, AIF-induced apoptosis is caspase-independent. The present inventors provide evidence herein that a novel AIF homologous molecule, AMID, can also induce caspase-independent apoptosis.

Specifically, this invention discloses a novel AIF homologous protein designated as AMID (AIF-homologous Mitochondrion-associated Inducer of Death). AMID is localized to the outer membrane of mitochondria. Overexpression of AMID induces caspase-independent apoptosis. Data suggests that AMID is induced by the tumor suppressor p53, although it may operate independently of p53 (see data below). In addition, expression of AMID is down-regulated in tumors compared to their individually matched normal counterparts (data not shown). AMID expression in the primary human tumors analyzed thus far demonstrated that in 19 out of 20, AMID was down-regulated. This suggests that AMID functions downstream of p53 control and plays a role in tumor suppression.

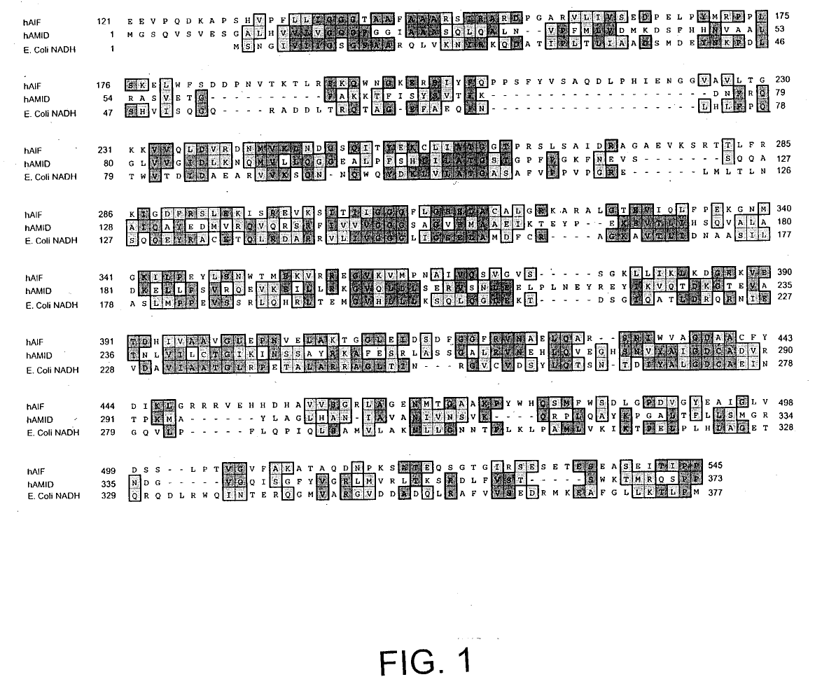

AMID is homologous to AIF protein (See FIG. 1). The experiments described herein demonstrate its ability to bind to the mitochondria and show that AMID is capable of inducing apoptosis by inducing its overexpression. Cell death occurs ˜30 hours after transfection of AMID in a dose-dependent manner into a cell line. The present invention includes a method to identify AMID and use this as a diagnostic indicator of the presence of cancer. An early diagnosis of cancer is critical to treat an individual and increase survivability. Furthermore, the present invention describes the use of the AMID protein and nucleic acid molecules in a therapeutic capacity for the treatment of tumors by targeted cell killing. In addition, this proposed treatment could be used in combination with other therapies to eradicate specific tumors and increase patient survival.

AIF is confined to the inter membrane space of mitochondria under physiological conditions. Upon certain apoptotic stimulation, AIF translocates into the nucleus and induces nuclear apoptosis by a unresolved mechanism. Although AMID shares significant homology with AIF, it lacks an N-terminal MLS. The immunofluorescent staining and biochemical experiments (see Examples) indicate that AMID is co-localized with mitochondria, most likely adhering to the outer membrane of mitochondria.

The present inventors have shown that overexpression of AMID induces cell death in a dose dependent manner. Like AIF, AMID-induced apoptosis is not inhibited by the pan-caspase inhibitor VAD-fmk or crmA, suggesting that AMID-induced apoptosis is caspase-independent. Interestingly, electronic microscopy studies show that AMID induces peripheral-type chromatin condensation, a phenotype that is very similar to that induced by AIF, but is in contrast to that induced by caspase-mediated apoptosis.

AMID-induced apoptosis is not inhibited by Bcl-2, indicating that AMID-induced apoptosis is not mediated by Bcl2 family member-induced mitochondrial permeability. Previously, it has been suggested that apoptosis induced by overexpression of a mitochondrion-targeting AIF precursor can be inhibited by Bcl-2. However, apoptosis induced by overexpression of cytosolic AIF lacking its MLS can not be inhibited by Bcl-2. In this context, AMID, which is associated with the outer membrane of mitochondria or localized in the cytosol, behaves similarly to the cytosolic AIF.

The domain mapping experiments presented in the Examples indicate that the C-terminal 187 aa (C-terminal 50%) of AMID is sufficient to induce apoptosis. The two N-terminal deletion mutants (aa77-373 and aa186-373 of SEQ ID NO:2), which are co-localized with mitochondria, induce apoptosis. Although no nuclear localization signal sequence is found in AMID, the two C-terminal deletion mutants (aa1-185 and aa1-300 of SEQ ID NO:2) are localized to the nucleus and can not induce apoptosis. The simplest explanation for these observations is that association with mitochondria is required for AMID-induced apoptosis.

The exact mechanism of how AMID induces apoptosis is still unknown. It is possible that association of AMID with mitochondria causes disruption of the mitochondrial structure and release of mitochondrial content and therefore, induction of apoptosis. One piece of evidence to support this hypothesis is the electronic microscopy observation of the present inventors that the AMID-induced apoptotic cells lack structurally preserved mitochondria. However, it is equally possible that AMID-induced apoptosis is independent of mitochondria and the loss of mitochondria in cells overexpressing AMID is a result of AMID-induced apoptosis.

Since AMID is homologous to AIF and also induces caspase-independent apoptosis, the inventors attempted to determine whether AMID and AIF are functionally connected. However, in contrast to previous reports, the present inventors' extensive efforts failed to demonstrate any apoptotic effects induced by overexpression of AIF or its cytosolic mutant lacking the MLS, whereas overexpression of AMID, as discussed above, was able to induce apoptosis.

The nucleic acid sequence encoding AMID that was identified by the present inventors was previously presented within, or partially by, several different expressed sequence tag clones from human sequences. The present inventors determined that one of these EST clones (Accession No. BG285370—a human cDNA clone derived from a prostate adenocarcinoma cell line) contained a coding region for the full length AMID protein, although this EST submission did not disclose a coding sequence within the clone, nor any proposed function for any protein that might be located within the clone. Another database submission (Accession No. AAH06121) disclosed a putative amino acid sequence encoded by another EST clone from renal cell adenocarcinoma (Accession No. BC006121) which has a sequence identical to the AMID amino acid sequence (SEQ ID NO:2). However, the protein was designated as an unknown protein and no function was proposed by the database submission.

The 373-amino acid, human AMID sequence described herein is represented by SEQ ID NO:2 (Accession No. NP_{—}116186, incorporated by reference in its entirety). SEQ ID NO:2 is approximately 86% identical to a mouse protein (Accession No. NP_{—}835159, incorporated herein by reference in its entirety), which published subsequent to the present invention, was designated as a product of RIKEN cDNA, and which appears to be a homologue of human AMID. The amino acid sequence for AMID is about 22% identical (44% homologous) over 218 amino acids to human AIF (Accession No. AAD16436, incorporated herein by reference in its entirety) and about 26% identical (48% homologous) over 233 amino acids to E. coli NADH oxidoreductase (Accession No. gi7449849, incorporated herein by reference in its entirety). FIG. 1 shows an alignment of SEQ ID NO:2, and relevant portions of AIF (SEQ ID NO:3) and E. coli NADH oxidoreductase (SEQ ID NO:4).

As used herein, reference to an isolated protein or polypeptide in the present invention, including an isolated AMID protein, includes full-length proteins, fusion proteins, or any fragment or homologue of such a protein. Such an AMID protein can include, but is not limited to, purified AMID protein, recombinantly produced AMID protein, membrane bound AMID protein, AMID protein complexed with lipids, soluble AMID protein and isolated AMID protein associated with other proteins. More specifically, an isolated protein, such as an AMID protein, according to the present invention, is a protein (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated AMID protein of the present invention is produced recombinantly. In addition, and by way of example, a “human AMID protein” refers to an AMID protein (generally including a homologue of a naturally occurring AMID protein) from a human (Homo sapiens) or to an AMID protein that has been otherwise produced from the knowledge of the structure (e.g., sequence) and perhaps the function of a naturally occurring AMID protein from Homo sapiens. In other words, a human AMID protein includes any AMID protein that has substantially similar structure and function of a naturally occurring AMID protein from Homo sapiens or that is a biologically active (i.e., has biological activity) homologue of a naturally occurring AMID protein from Homo sapiens as described in detail herein. As such, a human AMID protein can include purified, partially purified, recombinant, mutated/modified and synthetic proteins. According to the present invention, the terms “modification” and “mutation” can be used interchangeably, particularly with regard to the modifications/mutations to the amino acid sequence of AMID (or nucleic acid sequences) described herein. An isolated protein useful as an antagonist or agonist according to the present invention can be isolated from its natural source, produced recombinantly or produced synthetically.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either enhanced, decreased, or substantially similar properties as compared to the naturally occurring protein or peptide. A homologue can include an agonist of a protein or an antagonist of a protein.

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

Modifications in AMID homologues, as compared to the wild-type protein, either agonize, antagonize, or do not substantially change, the basic biological activity of the AMID homologue as compared to the naturally occurring protein, AMID. In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or mimetic (discussed below), may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein.

According to the present invention, an isolated AMID protein, including a biologically active homologue or fragment thereof, has at least one characteristic of biological activity of activity a wild-type, or naturally occurring AMID protein (which can vary depending on whether the homologue or fragment is an agonist, antagonist, or mimic of AMID). The biological activity of AMID can include, but is not limited to, induction of apoptosis in a cell, localization to the mitochondria (e.g., association with the outer membrane of mitochondrian), localization to the nucleus, induction of expression by p53, interaction with p53, oxidoreductase activity, and/or ADP-binding. Methods of detecting and measuring AMID expression and biological activity (and measuring AMID agonist or antagonist activity) include, but are not limited to, measurement of transcription of AMID, measurement of translation of AMID, measurement of cellular localization of AMID, measurement of binding or association of AMID with another protein, measurement of binding or association of AMID gene regulatory sequences to a protein or other nucleic acid, measurement of an increase or decrease in the induction of apoptosis in a cell that expresses AMID, measurement of ADP binding to AMID, measurement of oxidoreductase activity. It is noted that an isolated AMID protein of the present invention (including homologues) is not required to have AMID activity. An AMID protein can be a truncated, mutated or inactive protein, for example. In addition, an AMID protein that lacks apoptotic activity can be used as an oxidoreductase, and vice versa. Inactive AMID proteins are useful in diagnostic assays or some screening assays, for example, or for other purposes such as antibody production.

Methods to measure protein expression levels of AMID according to the invention include, but are not limited to: western blotting, immunocytochemistry, flow cytometry or other immunologic-based assays; assays based on a property of the protein including but not limited to, ligand binding or interaction with other protein partners. Binding assays are also well known in the art. For example, a BlAcore machine can be used to determine the binding constant of a complex between two proteins. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Other suitable assays for measuring the binding of one protein to another include, for example, immunoassays such as enzyme linked immunoabsorbent assays (ELISA) and radioimmunoassays (RIA), or determination of binding by monitoring the change in the spectroscopic or optical properties of the proteins through fluorescence, UV absorption, circular dichrosim, or nuclear magnetic resonance (NMR).

As used herein, the phrase “AMID agonist” refers to any compound that is characterized by the ability to agonize (e.g., stimulate, induce, increase, enhance, or mimic) the biological activity of a naturally occurring AMID as described herein. More particularly, an AMID agonist can include, but is not limited to, a protein, peptide, or nucleic acid that mimics or enhances the activity of the natural ligand, AMID, and includes any AMID homologue, binding protein (e.g., an antibody), agent that interacts with AMID, or any suitable product of drug/compound/peptide design or selection which is characterized by its ability to agonize (e.g., stimulate, induce, increase, enhance) the biological activity of a naturally occurring AMID protein in a manner similar to the natural agonist, AMID (e.g., by inducing apoptosis in a cell). Agonists of AMID of the present invention can be useful in methods for inducing apoptosis of cells for therapeutic purposes (e.g., tumor cells or HIV-infected cells) or in assays for identifying regulators of apoptosis, for example.

The phrase, “AMID antagonist” refers to any compound which inhibits (e.g., antagonizes, reduces, decreases, blocks, reverses, or alters) the effect of an AMID agonist as described above. More particularly, an AMID antagonist is capable of acting in a manner relative to AMID activity, such that the biological activity of the natural agonist AMID, is decreased in a manner that is antagonistic (e.g., against, a reversal of, contrary to) to the natural action of AMID. Such antagonists can include, but are not limited to, a protein, peptide, or nucleic acid (including ribozymes and antisense) or product of drug/compound/peptide design or selection that provides the antagonistic effect. Contact of an AMID ligand or a cell with an AMID antagonist, for example is useful for the inhibition of apoptosis or for the identification of regulators of apoptosis in an assay.

According to the present invention, a ribozyme typically contains stretches of complementary RNA bases that can base-pair with a target RNA ligand, including the RNA molecule itself, giving rise to an active site of defined structure that can cleave the bound RNA molecule (See Maulik et al., 1997, supra). Therefore, a ribozyme can serve as a targeting delivery vehicle for a nucleic acid molecule, or alternatively, the ribozyme can target and bind to RNA encoding AMID, for example, and thereby effectively inhibit the translation of AMID.

As used herein, an anti-sense nucleic acid molecule is defined as an isolated nucleic acid molecule that reduces expression of an AMID protein by hybridizing under high stringency conditions to a gene encoding the AMID protein. Such a nucleic acid molecule is sufficiently similar to the nucleic acid sequence encoding the AMID protein that the molecule is capable of hybridizing under high stringency conditions to the coding strand of the gene or RNA encoding the natural AMID protein. In a particularly preferred embodiment, an anti-sense nucleic acid molecule of the present invention is the exact complement of the coding region of an AMID protein or of a regulatory region of a gene encoding the AMID protein. It is noted that the anti-sense of the coding region does not necessarily include the anti-sense of the stop codon.

Homologues of AMID, including peptide and non-peptide agonists and antagonists of AMID, can be products of drug design or selection and can be produced using various methods known in the art. Such homologues can be referred to as mimetics. A mimetic refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art. Various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety.

A mimetic can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In one embodiment of the present invention, an AMID protein has an amino acid sequence that comprises, consists essentially of, or consists of, SEQ ID NO:2, amino acids 37-373 of SEQ ID NO:2, or amino acids 41-373 of SEQ ID NO:2. SEQ ID NO:2 represents a human AMID protein (encoded by nucleic acid sequence SEQ ID NO:1 and SEQ ID NO:5, with SEQ ID NO:5 representing only the coding sequence). Amino acids 37-373 or 41-373 represent truncated versions of AMID with alternate start sites, although the inventors' data and publications subsequent to the present invention indicate that position 1 of SEQ ID NO:2 is the start site. The present invention also includes homologues of SEQ ID NO:2, including fragments of SEQ ID NO:2 and sequences having a given identity to SEQ ID NO:2, wherein the homologue or fragment has AMID biological activity (including agonist or antagonist activity), as described previously herein.

In one embodiment, an AMID protein of the present invention, including an AMID homologue, comprises, consists essentially of, or consists of, an amino acid sequence that is at least about 45%, or at least about 50%, or at least about 55%, or at least about 60%, or at least about 65%, or at least about 70%, or at least about 75%, or at least about 80%, or at least about 85%, or at least about 90%, or at least about 95% identical, or at least about 95% identical, or at least about 96% identical, or at least about 97% identical, or at least about 98% identical, or at least about 99% identical, to the hAMID amino acid sequence represented by SEQ ID NO:2 over at least about 100 amino acids of SEQ ID NO:2, and more preferably over at least about 125 amino acids, and more preferably over at least about 150 amino acids, and more preferably over at least about 175 amino acids, and more preferably over at least about 200 amino acids, and more preferably over at least about 225 amino acids, and more preferably over at least about 250 amino acids, and more preferably over at least about 275 amino acids, and even more preferably over 300 amino acids, and more preferably over at least about 325 amino acids, and even more preferably over 350 amino acids, and even more preferably over the full length of the hAMID amino acid sequence represented by SEQ ID NO:2.

In one embodiment, an AMID protein of the present invention comprises an amino acid sequence that is less than 100% identical to SEQ ID NO:2 (i.e., an AMID homologue). In another aspect of the invention, an AMID homologue according to the present invention has an amino acid sequence that is less than about 99% identical to SEQ ID NO:2, and in another embodiment, is less than 98% identical to SEQ ID NO:2, and in another embodiment, is less than 97% identical to SEQ ID NO:2, and in another embodiment, is less than 96% identical to SEQ ID NO:2, and in another embodiment, is less than 95% identical to SEQ ID NO:2, and in another embodiment, is less than 94% identical to SEQ ID NO:2, and in another embodiment, is less than 93% identical to SEQ ID NO:2, and in another embodiment, is less than 92% identical to SEQ ID NO:2, and in another embodiment, is less than 91% identical to SEQ ID NO:2, and in another embodiment, is less than 90% identical to SEQ ID NO:2, and so on, in increments of whole integers. The isolated AMID protein of the present invention, including an AMID homologue, preferably has AMID biological activity.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schaaffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on) For blastp, using 0 BLOSUM62 matrix:

Open gap (11) and extension gap (1) penalties gap x_dropoff (50) expect (10) word size (3) filter (on).

An AMID protein can also include proteins having an amino acid sequence comprising at least 10 contiguous amino acid residues of SEQ ID NO:2 (i.e., 10 contiguous amino acid residues having 100% identity with 10 contiguous amino acids of SEQ ID NO:2). Even small fragments of AMID without AMID biological activity are useful in the present invention, for example, in the preparation of antibodies against the full-length protein. In other embodiments, a homologue of an AMID amino acid sequence includes amino acid sequences comprising at least 20, or at least 30, or at least 40, or at least 50, or at least 75, or at least 100, or at least 125, or at least 150, or at least 175, or at least 150, or at least 200, or at least 250, or at least 300, or at least 350, or at least 373 contiguous amino acid residues of the amino acid sequence represented by SEQ ID NO:2. In a preferred embodiment, an AMID homologue has measurable or detectable AMID biological activity.

According to the present invention, the term “contiguous” or “consecutive”, with regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

One of skill in the art will be able to readily produce and identify AMID homologues according to the invention, and particularly, AMID homologues having AMID biological activity. The present inventors have provided an alignment of AMID and other related NADH-oxidoreductases/flavoproteins (i.e., AIF and E. Coli NADH oxidoreductase) in FIG. 1, which illustrates amino acids that are conserved among the proteins. This alignment provides one of skill in the art with guidance as to which amino acids will be most likely to tolerate change in the production of AMID homologues of the invention. In one embodiment of the invention, an AMID protein includes any fragment of an AMID protein, and particularly a biologically active fragment (i.e., a fragment that has AMID activity, such as the ability to induce apoptosis in a cell). The present inventors have also demonstrated through the production of AMID deletion mutants, that as much as 50% of the N-terminus of AMID may be deleted and result in an AMID homologue that is associated with the mitochondrian and retains the ability to induce apoptosis. Preferred AMID fragments of the invention include any fragments of SEQ ID NO:2 when at least 1 amino acid from the N-terminus is deleted (e.g., 2-373 of SEQ ID NO:2), up to at least 185 amino acids of the N-terminal amino acids are deleted (e.g., 186-373 of SEQ ID NO:2), and includes intermediate fragments (e.g., 3-373, 8-37-, 34-373, 87-373 of SEQ ID NO:2, etc.). Exemplary fragments include, but are not limited to: a fragment consisting essentially of amino acids 77-373 of SEQ ID NO:2 or amino acids 186-373 of SEQ ID NO:2. Other fragments having AMID biological activity will be clearly envisioned by those of skill in the art given the guidance provided herein. Fragments having C-terminal deletions (e.g., 1-185 or 1-300 of SEQ ID NO:2) have been found to lack apoptosis-inducing activity, but are localized to the nucleus and contain sequence necessary for oxidoreductase enzymatic activity. As such, these fragments have a nucleus-localizing activity of AMID and may be useful, for example, to deliver other proteins or compounds to the nucleus. Alternatively, such fragments are useful as oxidoreductases.

In another embodiment, an AMID protein, including an AMID homologue, includes a protein having an amino acid sequence that is sufficiently similar to a natural AMID amino acid sequence that a nucleic acid sequence encoding the homologue is capable of hybridizing under moderate, high or very high stringency conditions (described below) to (i.e., with) a nucleic acid molecule encoding the natural AMID protein (i.e., to the complement of the nucleic acid strand encoding the natural AMID amino acid sequence). Preferably, a homologue of an AMID protein is encoded by a nucleic acid molecule comprising a nucleic acid sequence that hybridizes under low, moderate, or high stringency conditions to the complement of a nucleic acid sequence that encodes a protein comprising an amino acid sequence represented by SEQ ID NO:2. Such hybridization conditions are described in detail below.

A nucleic acid sequence complement of nucleic acid sequence encoding an AMID protein of the present invention refers to the nucleic acid sequence of the nucleic acid strand that is complementary to the strand which encodes AMID. It will be appreciated that a double stranded DNA which encodes a given amino acid sequence comprises a single strand DNA and its complementary strand having a sequence that is a complement to the single strand DNA. As such, nucleic acid molecules of the present invention can be either double-stranded or single-stranded, and include those nucleic acid molecules that form stable hybrids under stringent hybridization conditions with a nucleic acid sequence that encodes an amino acid sequence such as SEQ ID NO:2, and/or with the complement of the nucleic acid sequence that encodes an amino acid sequence such as SEQ ID NO:2. Methods to deduce a complementary sequence are known to those skilled in the art. It should be noted that since nucleic acid sequencing technologies are not entirely error-free, the sequences presented herein, at best, represent apparent sequences of an AMID protein of the present invention.

As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na+) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na+) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC). AMID proteins also include expression products of gene fusions (for example, used to overexpress soluble, active forms of the recombinant protein), of mutagenized genes (such as genes having codon modifications to enhance gene transcription and translation), and of truncated genes (such as genes having membrane binding domains removed to generate soluble forms of a membrane protein, or genes having signal sequences removed which are poorly tolerated in a particular recombinant host).

The minimum size of a protein and/or homologue of the present invention is a size sufficient to have AMID biological activity or, when the protein is not required to have such activity, sufficient to be useful for another purpose associated with an AMID protein of the present invention, such as for the production of antibodies that bind to a naturally occurring AMID protein. In one embodiment, the AMID protein of the present invention is at least 20 amino acids in length, or at least about 25 amino acids in length, or at least about 30 amino acids in length, or at least about 40 amino acids in length, or at least about 50 amino acids in length, or at least about 60 amino acids in length, or at least about 70 amino acids in length, or at least about 80 amino acids in length, or at least about 90 amino acids in length, or at least about 100 amino acids in length, or at least about 125 amino acids in length, or at least about 150 amino acids in length, or at least about 175 amino acids in length, or at least about 200 amino acids in length, or at least about 250 amino acids in length, and so on up to a full length AMID protein, and including any size in between 20 and 373 amino acids in increments of one whole integer (one amino acid). There is no limit, other than a practical limit, on the maximum size of such a protein in that the protein can include a portion of an AMID protein or a full-length AMID protein, plus additional sequence (e.g., a fusion protein sequence), if desired.

Similarly, the minimum size of a nucleic acid molecule of the present invention is a size sufficient to encode a protein having AMID activity, sufficient to encode a protein comprising at least one epitope which binds to an antibody, or sufficient to form a probe or oligonucleotide primer that is capable of forming a stable hybrid with the complementary sequence of a nucleic acid molecule encoding a natural AMID protein (e.g., under moderate, high, or high stringency conditions). As such, the size of the nucleic acid molecule encoding such a protein can be dependent on nucleic acid composition and percent homology or identity between the nucleic acid molecule and complementary sequence as well as upon hybridization conditions per se (e.g., temperature, salt concentration, and formamide concentration). The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a portion of an AMID protein encoding sequence, a nucleic acid sequence encoding a full-length AMID protein (including a AMID protein gene), including any length fragment between about 20 and 1119 nucleotides, in whole integers (e.g., 20, 21, 22, 23, 24, 25 . . . 1118, 1119 nucleotides), or multiple genes, or portions thereof.

The present invention also includes a fusion protein that includes an AMID protein-containing domain (including a homologue of AMID) attached to one or more fusion segments, which are typically heterologous in sequence to the AMID sequence (i.e., different than AMID sequence). Suitable fusion segments for use with the present invention include, but are not limited to, segments that can: enhance a protein's stability; provide other desirable biological activity; and/or assist with the purification of AMID (e.g., by affinity chromatography). A suitable fision segment can be a domain of any size that has the desired function (e.g., imparts increased stability, solubility, action or biological activity; and/or simplifies purification of a protein). Fusion segments can bejoined to amino and/or carboxyl termini of the AMID protein-containing domain of the protein and can be susceptible to cleavage in order to enable straight-forward recovery of the protein. Fusion proteins are preferably produced by culturing a recombinant cell transfected with a fusion nucleic acid molecule that encodes a protein including the fusion segment attached to either the carboxyl and/or amino terminal end of a AMID protein-containing domain.

In one embodiment of the present invention, any of the amino acid sequences described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

Another embodiment of the present invention relates to a composition comprising at least about 500 ng, and preferably at least about 1 μg, and more preferably at least about 5 μg, and more preferably at least about 10 μg, and more preferably at least about 25 μg, and more preferably at least about 50 μg, and more preferably at least about 75 μg, and more preferably at least about 100 μg, and more preferably at least about 250 μg, and more preferably at least about 500 μg, and more preferably at least about 750 μg, and more preferably at least about 1 mg, and more preferably at least about 5 mg, of an isolated AMID protein comprising any of the AMID proteins or homologues thereof discussed herein. Such a composition of the present invention can include any carrier with which the protein is associated by virtue of the protein preparation method, a protein purification method, or a preparation of the protein for use in an in vitro, ex vivo, or in vivo method according to the present invention. For example, such a carrier can include any suitable excipient, buffer and/or delivery vehicle, such as a pharmaceutically acceptable carrier (discussed below), which is suitable for combining with the AMID protein of the present invention so that the protein can be used in vitro, ex vivo or in vivo according to the present invention.

Further embodiments of the present invention include nucleic acid molecules that encode an AMID protein. A nucleic acid molecule of the present invention includes a nucleic acid molecule comprising, consisting essentially of, or consisting of, a nucleic acid sequence encoding any of the isolated AMID proteins, including an AMID homologue, described above. In a preferred embodiment a nucleic molecule of the present invention includes a nucleic acid molecule comprising, consisting essentially of, or consisting of, a nucleic acid sequence represented by SEQ ID NO:1, SEQ ID NO:5, or fragments or homologues thereof encoding an AMID protein useful in the invention, or encompassing useful oligonucleotides and complementary nucleic acid sequences.

In one embodiment, such nucleic acid molecules include isolated nucleic acid molecules that hybridize under moderate stringency conditions, and more preferably under high stringency conditions, and even more preferably under very high stringency conditions, as described above, with the complement of a nucleic acid sequence encoding a naturally occurring AMID protein (i.e., including naturally occurring allelic variants encoding an AMID protein). Preferably, an isolated nucleic acid molecule encoding an AMID protein of the present invention comprises a nucleic acid sequence that hybridizes under moderate, high, or very high stringency conditions to the complement of a nucleic acid sequence that encodes a protein comprising an hAMID amino acid sequence represented by SEQ ID NO:2.

In accordance with the present invention, an isolated nucleic acid molecule is a nucleic acid molecule (polynucleotide) that has been removed from its natural milieu (i.e., that has been subject to human manipulation) and can include DNA, RNA, or derivatives of either DNA or RNA, including cDNA. As such, “isolated” does not reflect the extent to which the nucleic acid molecule has been purified. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. An isolated AMID nucleic acid molecule of the present invention can be isolated from its natural source or produced using recombinant DNA technology (e.g., polymerase chain reaction (PCR) amplification, cloning) or chemical synthesis. Isolated AMID nucleic acid molecules can include, for example, AMID genes, natural allelic variants of AMID genes, AMID coding regions or portions thereof, and AMID coding and/or regulatory regions modified by nucleotide insertions, deletions, substitutions, and/or inversions in a manner such that the modifications do not substantially interfere with the nucleic acid molecule's ability to encode an AMID protein of the present invention or to form stable hybrids under stringent conditions with natural gene isolates. An isolated AMID nucleic acid molecule can include degeneracies. As used herein, nucleotide degeneracies refers to the phenomenon that one amino acid can be encoded by different nucleotide codons. Thus, the nucleic acid sequence of a nucleic acid molecule that encodes an AMID protein of the present invention can vary due to degeneracies. It is noted that an AMID nucleic acid molecule of the present invention is not required to encode a protein having AMID protein activity. An AMID nucleic acid molecule can encode a truncated, mutated or inactive protein, for example. Such nucleic acid molecules and the proteins encoded by such nucleic acid molecules are useful in as probes and primers for the identification of other AMID proteins. If the nucleic acid molecule is an oligonucleotide, such as a probe or primer, the oligonucleotide preferably ranges from about 5 to about 50 or about 500 nucleotides, more preferably from about 10 to about 40 nucleotides, and most preferably from about 15 to about 40 nucleotides in length.

According to the present invention, reference to an AMID gene includes all nucleic acid sequences related to a natural (i.e. wild-type) AMID gene, such as regulatory regions that control production of the AMID protein encoded by that gene (such as, but not limited to, transcription, translation or post-translation control regions) as well as the coding region itself. In another embodiment, an AMID gene can be a naturally occurring allelic variant that includes a similar but not identical sequence to the nucleic acid sequence encoding a given AMID protein. Allelic variants have been previously described above. The phrases “nucleic acid molecule” and “gene” can be used interchangeably when the nucleic acid molecule comprises a gene as described above.

Preferably, an isolated nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on protein biological activity. Allelic variants and protein homologues (e.g., proteins encoded by nucleic acid homologues) have been discussed in detail above.

An AMID nucleic acid molecule homologue (i.e., encoding an AMID protein homologue) can be produced using a number of methods known to those skilled in the art (see, for example, Sambrook et al.). For example, nucleic acid molecules can be modified using a variety of techniques including, but not limited to, by classic mutagenesis and recombinant DNA techniques (e.g., site-directed mutagenesis, chemical treatment, restriction enzyme cleavage, ligation of nucleic acid fragments and/or PCR amplification), or synthesis of oligonucleotide mixtures and ligation of mixture groups to “build” a mixture of nucleic acid molecules and combinations thereof. Another method for modifying a recombinant nucleic acid molecule encoding an AMID protein is gene shuffling (i.e., molecular breeding) (See, for example, U.S. Pat. No. 5,605,793 to Stemmer; Minshull and Stemmer; 1999, Curr. Opin. Chem. Biol. 3:284-290; Stemmer, 1994, P.N.A.S. USA 91:10747-10751, all of which are incorporated herein by reference in their entirety). This technique can be used to efficiently introduce multiple simultaneous changes in the AMID protein. Nucleic acid molecule homologues can be selected by hybridization with an AMID gene or by screening the function of a protein encoded by a nucleic acid molecule (i.e., biological activity).

In one embodiment, an oligonucleotide of the present invention, a plurality of oligonucleotides of the present invention, or other nucleic acids of the invention, are immobilized on a substrate, such as for use in a screening assay. In general, an array, an oligonucleotide, a cDNA, or genomic DNA, that is a portion of a gene (e.g., a gene encoding AMID) occupies a known location on a substrate (e.g., is immobilized on a substrate). A nucleic acid target sample is hybridized with the immobilized nucleic acid and then the amount of target nucleic acids hybridized to each probe in the array/assay is quantified. One preferred quantifying method is to use confocal microscope and fluorescent labels. The Affymetrix GeneChip™ Array system (Affymetrix, Santa Clara, Calif.) and the Atlas™ Human cDNA Expression Array system are particularly suitable for quantifying the hybridization; however, it will be apparent to those of skill in the art that any similar systems or other effectively equivalent detection methods can also be used. In a particularly preferred embodiment, one can use the knowledge of the gene described herein to design novel arrays of polynucleotides, cDNAs or genomic DNAs for screening methods (e.g., cancer diagnostics) described herein. Such novel pluralities of polynucleotides are contemplated to be a part of the present invention.

In one embodiment, the polynucleotide probes are conjugated to detectable markers. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Preferably, the polynucleotide probes are immobilized on a substrate such as: artificial membranes, organic supports, biopolymer supports and inorganic supports.

Suitable nucleic acid samples for screening on an array contain transcripts of interest or nucleic acids derived from the transcripts of interest (i.e., transcripts derived from the PR-regulated genes of the present invention). As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like.

One embodiment of the present invention relates to a recombinant nucleic acid molecule which comprises the isolated nucleic acid molecule described above which is operatively linked to at least one transcription control sequence. More particularly, according to the present invention, a recombinant nucleic acid molecule typically comprises a recombinant vector and the isolated nucleic acid molecule as described herein. According to the present invention, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is, nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant host cell, although it is preferred if the vector remain separate from the genome for most applications of the invention. The entire vector can remain in place within a host cell, or under certain conditions, the plasmid DNA can be deleted, leaving behind the nucleic acid molecule of the present invention. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one embodiment, a recombinant vector used in a recombinant nucleic acid molecule of the present invention is an expression vector. As used herein, the phrase “expression vector” is used to refer to a vector that is suitable for production of an encoded product (e.g., a protein of interest). In this embodiment, a nucleic acid sequence encoding the product to be produced (e.g., the AMID protein or homologue thereof) is inserted into the recombinant vector to produce a recombinant nucleic acid molecule. The nucleic acid sequence encoding the protein to be produced is inserted into the vector in a manner that operatively links the nucleic acid sequence to regulatory sequences in the vector which enable the transcription and translation of the nucleic acid sequence within the recombinant host cell.

In another embodiment of the invention, the recombinant nucleic acid molecule comprises a viral vector. A viral vector includes an isolated nucleic acid molecule of the present invention integrated into a viral genome or portion thereof, in which the nucleic acid molecule is packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

Typically, a recombinant nucleic acid molecule includes at least one nucleic acid molecule of the present invention operatively linked to one or more transcription control sequences. As used herein, the phrase “recombinant molecule” or “recombinant nucleic acid molecule” primarily refers to a nucleic acid molecule or nucleic acid sequence operatively linked to a transcription control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to a transcription control sequence in a manner such that the molecule is able to be expressed when transfected (i.e., transformed, transduced, transfected, conjugated or conduced) into a host cell. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell or organism into which the recombinant nucleic acid molecule is to be introduced.

Recombinant nucleic acid molecules of the present invention can also contain additional regulatory sequences, such as translation regulatory sequences, origins of replication, and other regulatory sequences that are compatible with the recombinant cell. In one embodiment, a recombinant molecule of the present invention, including those which are integrated into the host cell chromosome, also contains secretory signals (i.e., signal segment nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another embodiment, a recombinant molecule of the present invention comprises a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells or plants. In microbial systems, the term “transformation” is used to describe an inherited change due to the acquisition of exogenous nucleic acids by the microorganism and is essentially synonymous with the term “transfection.” However, in animal cells, transformation has acquired a second meaning which can refer to changes in the growth properties of cells in culture (described above) after they become cancerous, for example. Therefore, to avoid confusion, the term “transfection” is preferably used with regard to the introduction of exogenous nucleic acids into animal cells, and is used herein to generally encompass transfection of animal cells and transformation of plant cells and microbial cells, to the extent that the terms pertain to the introduction of exogenous nucleic acids into a cell. Therefore, transfection techniques include, but are not limited to, transformation, particle bombardment, electroporation, microinjection, lipofection, adsorption, infection and protoplast fusion.

One or more recombinant molecules of the present invention can be used to produce an encoded product (e.g., AMID protein) of the present invention. In one embodiment, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include, but are not limited to, any bacterial, fungal (e.g., yeast), insect, or animal cell that can be transfected. Host cells can be either untransfected cells or cells that are already transfected with at least one other recombinant nucleic acid molecule.

In one embodiment, one or more protein(s) expressed by an isolated nucleic acid molecule of the present invention are produced by culturing a cell that expresses the protein (i.e., a recombinant cell or recombinant host cell) under conditions effective to produce the protein. In some instances, the protein may be recovered, and in others, the cell may be harvested in whole (e.g., for ex vivo administration), either of which can be used in a composition. A preferred cell to culture is any suitable host cell as described above. Effective culture conditions include, but are not limited to, effective media, bioreactor, temperature, pH and oxygen conditions that permit protein production and/or recombination. An effective medium refers to any medium in which a given host cell is typically cultured. Such medium typically comprises an aqueous medium having assimilable carbon, nitrogen and phosphate sources, and appropriate salts, minerals, metals and other nutrients, such as vitamins. Cells can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the culture medium; be secreted into a space between two cellular membranes; or be retained on the outer surface of a cell membrane. The phrase “recovering the protein” refers to collecting the whole culture medium containing the protein and need not imply additional steps of separation or purification. Proteins produced according to the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Proteins of the present invention are preferably retrieved, obtained, and/or used in “substantially pure” form. As used herein, “substantially pure” refers to a purity that allows for the effective use of the protein in vitro, ex vivo or in vivo according to the present invention. For a protein to be useful in an in vitro, ex vivo or in vivo method according to the present invention, it is substantially free of contaminants, other proteins and/or chemicals that might interfere or that would interfere with its use in a method disclosed by the present invention, or that at least would be undesirable for inclusion with an AMID protein (including homologues) when it is used in a method disclosed by the present invention. Such methods include antibody production, agonist/antagonist identification assays, preparation of therapeutic compositions, administration in a therapeutic composition, and all other methods disclosed herein. Preferably, a “substantially pure” protein, as referenced herein, is a protein that can be produced by any method (i.e., by direct purification from a natural source, recombinantly, or synthetically), and that has been purified from other protein components such that the protein comprises at least about 80% weight/weight of the total protein in a given composition (e.g., the AMID protein is about 80% of the protein in a solution/composition/buffer), and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99%, weight/weight of the total protein in a given composition.

It will be appreciated by one skilled in the art that use of recombinant DNA technologies can improve control of expression of transfected nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter. Recombinant techniques useful for controlling the expression of nucleic acid molecules include, but are not limited to, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules to correspond to the codon usage of the host cell, and deletion of sequences that destabilize transcripts.

Another embodiment of the present invention relates to an isolated binding agent selected from an antibody, an antigen binding fragment, or a binding partner. The binding agent selectively binds to an amino acid sequence selected from SEQ ID NO:2, including to any fragment of SEQ ID NO:2 comprising at least one antibody binding epitope. According to the present invention, the phrase “selectively binds to” refers to the ability of an antibody, antigen binding fragment or binding partner of the present invention to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

Antibodies are characterized in that they comprise immunoglobulin domains and as such, they are members of the immunoglobulin superfamily of proteins. An antibody of the invention includes polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Genetically engineered antibodies of the invention include those produced by standard recombinant DNA techniques involving the manipulation and re-expression of DNA encoding antibody variable and/or constant regions. Particular examples include, chimeric antibodies, where the VH and/or VL domains of the antibody come from a different source to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

The invention also extends to non-antibody polypeptides, sometimes referred to as binding partners, that have been designed to bind specifically to, and either activate or inhibit as appropriate, an AMID protein of the invention. Examples of the design of such polypeptides, which possess a prescribed ligand specificity are given in Beste et al. (Proc. Natl. Acad. Sci. 96:1898-1903, 1999), incorporated herein by reference in its entirety.

In one embodiment, a binding agent of the invention is immobilized on a substrate such as: artificial membranes, organic supports, biopolymer supports and inorganic supports such as for use in a screening assay.

The AMID proteins (including homologues), as well as nucleic acids encoding such proteins, antibodies that bind to such proteins, and other tools related to AMID described herein, can be used in a variety of methods. For example, one embodiment of the present invention relates to a method to diagnose a disorder associated with AMID expression or biological activity. The method includes the steps of detecting expression or biological activity of AMID or a nucleic acid encoding AMID in a tissue or cells of a patient suspected of having the disorder, and comparing the expression or biological activity to a control, wherein a difference in the expression or biological activity of AMID or a nucleic acid encoding AMID in a tissue or cells of the patient as compared to the control indicates a positive diagnosis of a disorder associated with AMID. In particular, AMID expression is useful in the present invention to detect the presence of a cancer in a patient (e.g., to identify a tumor cell in a patient). More specifically, the present inventors have found that expression of wild-type (normal) AMID is lower in most cancer cells tested as compared to their normal counterpart cells (wild-type, non-cancerous, non-transformed cells). In some cancer cells, one may also be able to detect high levels of expression of a mutated AMID. This would be a similar phenomenon as occurs with p53, which is a tumor suppressor gene. When p53 is deleted, cells are more easily transformed. However, some tumors have high levels of mutated p53 (point mutation), such that upon the initial discovery of p53, it was thought that this protein was overexpressed in tumors, until the identity of the mutant was discovered. Without being bound by theory, the present inventors consider that cancer cells might also overexpress a mutated form of AMID, while in general, wild-type AMID will be downregulated in tumor cells as compared to normal (non-cancer) cells of the same type. Such mutated AMID forms may contribute to tumorigenesis (e.g., via reduced suppression of tumor growth by reduced apoptotic activity in the cell). Therefore, in one embodiment, a method of detecting cancer includes detecting a mutated AMID which is expressed at low, normal or higher levels than wild-type AMID and as compared to a normal control cell.

Therefore, one embodiment of the invention is a method for detecting the presence of a cancer in a patient, comprising the steps of: (a) obtaining a biological sample comprising cells from a patient; (b) detecting in the cells the expression of a protein comprising SEQ ID NO:2 or a nucleic acid molecule comprising SEQ ID NO:5; and (c) comparing the level of expression of the protein or nucleic acid molecule detected in (b) to a control expression level as an indicator of the presence of a cancer in the patient, wherein a decrease in the expression of the protein or the nucleic acid molecule as compared to the control is an indicator of a positive diagnosis of cancer in the patient. In one embodiment, the step of detecting comprises contacting the biological sample with a binding agent that binds to a protein comprising SEQ ID NO:2, and detecting in the sample expression of a protein that binds to the binding agent. For example, such a binding agent can be chosen from: an antibody, an antigen binding fragment, and a peptide that selectively binds to SEQ ID NO:2. In one embodiment, the step of contacting comprises contacting the cells in the biological sample intracellularly with the binding agent. Since AMID is an intracellular molecule, typically, either cells are lysed to release the protein, or the contacting agent is delivered into the cell. In one embodiment, the step of detecting comprises contacting nucleic acids in the biological sample with an oligonucleotide consisting essentially of at least 22 consecutive nucleotides of SEQ ID NO: 5, or the complement thereof, and detecting in the sample nucleic acids that hybridize to the oligonucleotide under highly stringent conditions. In one aspect, the step of detecting comprises detecting expression of an RNA sequence comprising SEQ ID NO:5 or a fragment thereof in the biological sample as compared to expression of the RNA sequence in the control. In another aspect, the step of detecting comprises detecting a nucleic acid sequence in a sample comprising a cDNA product of RNA comprising SEQ ID NO:5 or a fragment thereof as compared to a cDNA product of RNA in the control. In another aspect, the detecting comprises detecting a nucleic acid sequence in a sample comprising amplified nucleic acid products of RNA comprising SEQ ID NO:5 or a fragment thereof in the sample as compared to amplified nucleic acid products of RNA from the control. Preferably, a statistically significant deviation from the control amount indicates the presence of cancer in the patient.

In another aspect, the method can include a step of detecting a mutated AMID protein or mutated AMID-encoding nucleic acid molecule in the cell, such as by hybridization, amplification and/or sequencing AMID or AMID-encoding nucleic acids in the cell and comparing the AMID or AMID encoding-nucleic acids to the wild-type AMID protein or nucleic acid sequence disclosed herein. Detection of a mutated AMID, and particularly, an AMID protein or nucleic acid molecule expressed at high levels as compared to a normal control, can also be an indicator of a positive diagnosis of cancer.

The terms “diagnose”, “diagnosis”, “diagnosing” and variants thereof refer to the identification of a disease or condition on the basis of its signs and symptoms. As used herein, a “positive diagnosis” indicates that the disease or condition, or a potential for developing the disease or condition, has been identified. In contrast, a “negative diagnosis” indicates that the disease or condition, or a potential for developing the disease or condition, has not been identified.

According to the present invention, the term “cell sample” can be used generally to refer to a sample of any type which contains cells to be evaluated by the present method, including but not limited to, a sample of isolated cells, a tissue sample and/or a bodily fluid sample. According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells which are preferably evaluated. Cells can be obtained, for example, by scraping of a tissue, processing of a tissue sample to release individual cells, or isolation from a bodily fluid. A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch. A bodily fluid sample, like the tissue sample, contains the cells to be evaluated for AMID or amid expression or biological activity, and is a fluid obtained by any method suitable for the particular bodily fluid to be sampled. Bodily fluids suitable for sampling include, but are not limited to, blood, mucous, seminal fluid, saliva, breast milk, bile and urine.

Methods suitable for detecting amid transcription or AMID translation are described elsewhere herein and include any suitable method for detecting and/or measuring mRNA levels or protein levels from a cell, cell extract or tissue. Methods for detecting transcription include, but are not limited to: polymerase chain reaction (PCR), reverse transcriptase PCR (RT-PCR), in situ hybridization, Northern blot, sequence analysis, and detection of a reporter gene. Such methods for detection of transcription levels are well known in the art, and many of such methods are described in Sambrook et al., Molecular Cloning: A Laboratory Manual, Cold Spring Harbor Labs Press, 1989 and/or in Glick et al., Molecular Biotechnology: Principles and Applications of Recombinant DNA, ASM Press, 1998; Sambrook et al., ibid., and Glick et al., ibid. are incorporated by reference herein in their entireties. Methods suitable for the detection of AMID protein include, but are not limited to, immunoblot (e.g., Western blot), enzyme-linked immunosorbant assay (ELISA), radioimmunoassay (RIA), immunoprecipitation, immunohistochemistry and immunofluorescence. Such methods are well known in the art.

The method of the present invention includes a step of comparing the level of AMID or amid expression or biological activity detected in step (a) to a baseline level of AMID or amid expression or biological activity. According to the present invention, a “baseline level” is a control level, and in some embodiments, a normal level of AMID or amid expression or activity (e.g., from a normal patient sample, a non-cancerous cell, a preestablished baseline, etc.) against which a test level of AMID or amid expression or biological activity (i.e., in the patient sample) can be compared. The method for establishing a baseline level of AMID or amid expression or activity is selected based on the sample type, the tissue or organ from which the sample is obtained, the status of the patient to be evaluated, and the focus or goal of the assay (e.g., diagnosis, staging, monitoring). Preferably, the method is the same method that will be used to evaluate the sample in the patient. Baseline levels can be established using an autologous control sample obtained from the patient, an autologous level in a previous sample from the same patient, or using presestablished control samples that were obtained from a population of matched individuals.

One embodiment of the invention relates to a diagnostic kit useful for detecting the presence of cancer in a patient. The diagnostic kit can include at least one oligonucleotide or other nucleic acid molecule according to the present invention and/or at least one antibody or binding protein that selectively binds to an AMID protein. The kits can include detection agents, including labels for the oligonucleotide and/or antibodies, or other secondary detection reagents.

Some embodiments of the present invention include a composition comprising AMID or a regulator thereof for diagnostic, screening or therapeutic purposes. Therefore, another embodiment of the invention relates to a composition comprising a compound selected from: (i) an isolated AMID protein, fragment or homologue thereof (including agonists and antagonists that are proteins); (ii) an AMID agonist or antagonist compound other than a protein AMID homologue (e.g., a product of drug design); or (iii) an isolated antibody that specifically binds to an AMID protein of (i). The composition typically also includes a pharmaceutically acceptable carrier. In this aspect of the present invention, an isolated AMID protein can be any of the AMID proteins previously described herein, including, but not limited to, a wild-type AMID protein, an AMID protein homologue, a fragment of AMID and/or an AMID fusion protein. An isolated antibody that selectively binds to an AMID protein has also been described above. Agonists and antagonists of AMID have also been described above. In one embodiment, a composition of the present invention can include nucleic acid molecules encoding AMID or a homologue, fragment or fusion protein of AMID. In one embodiment, a composition of the present invention includes a combination of at least two of any of the above-identified compounds. The compositions and their components can be used in any of the diagnostic or therapeutic embodiments of the invention described herein.

Compositions of the present invention are useful for regulating biological processes and particularly, processes associated with apoptosis. Such processes typically involve tumor cells. In particular, compositions of the present invention are useful for regulating the expression and/or biological activity of AMID, including the interaction or association of AMID with other proteins or nucleic acids, as well as the induction of apoptosis in a cell. In some embodiments, such compositions are useful for increasing (e.g., costimulating, enhancing, upregulating) the expression and/or biological activity of AMID. In some embodiments, such compositions are useful for decreasing (e.g., inhibiting, reducing, downregulating) the expression and/or biological activity of AMID.

According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. A suitable in vitro, in vivo or ex vivo site is preferably any site where it is desirable to regulate apoptosis. In one embodiment, a suitable site is a the site of a tumor. Preferred pharmaceutically acceptable carriers are capable of maintaining a protein, compound, or recombinant nucleic acid molecule of the present invention in a form that, upon arrival of the protein, compound, or recombinant nucleic acid molecule at the cell target in a culture or in patient, the protein, compound or recombinant nucleic acid molecule is capable of interacting with its target (e.g., a naturally occurring AMID protein, an amid gene, an AMID ligand, etc.).

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Compositions of the present invention can be sterilized by conventional methods and/or lyophilized.

One type of pharmaceutically acceptable carrier includes a controlled release formulation that is capable of slowly releasing a composition of the present invention into a patient or culture. As used herein, a controlled release formulation comprises a compound of the present invention (e.g., a protein (including homologues), an antibody, a nucleic acid molecule, or a mimetic) in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to a patient, form a solid or a gel in situ. Preferred carriers are also biodegradable (i.e., bioerodible). When the compound is a recombinant nucleic acid molecule, suitable carriers include, but are not limited to liposomes, viral vectors or other carriers, including ribozymes, gold particles, poly-L-lysine/DNA-molecular conjugates, and artificial chromosomes. Natural lipid-containing carriers include cells and cellular membranes. Artificial lipid-containing carriers include liposomes and micelles.

A carrier of the present invention can be modified to target to a particular site in a patient, thereby targeting and making use of a compound of the present invention at that site. A pharmaceutically acceptable carrier which is capable of targeting can also be referred to herein as a “delivery vehicle” or “targeting carrier”. Suitable modifications include manipulating the chemical formula of the lipid portion of the delivery vehicle and/or introducing into the vehicle a targeting agent capable of specifically targeting a delivery vehicle to a preferred site or target site, for example, a preferred cell type. A “target site” refers to a site in a patient to which one desires to deliver a composition. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

One preferred delivery vehicle of the present invention is a liposome. A liposome is capable of remaining stable in an animal for a sufficient amount of time to deliver a nucleic acid molecule described in the present invention to a preferred site in the animal. A liposome, according to the present invention, comprises a lipid composition that is capable of delivering a nucleic acid molecule described in the present invention to a particular, or selected, site in a patient. A liposome according to the present invention comprises a lipid composition that is capable of fusing with the plasma membrane of the targeted cell to deliver a nucleic acid molecule into a cell. Liposomes are particularly useful carriers for regulatory molecules of the present invention, since AMID is an intracellular molecule. Suitable liposomes for use with the present invention include any liposome. Preferred liposomes of the present invention include those liposomes commonly used in, for example, gene delivery methods known to those of skill in the art. More preferred liposomes comprise liposomes having a polycationic lipid composition and/or liposomes having a cholesterol backbone conjugated to polyethylene glycol. Complexing a liposome with a nucleic acid molecule or protein of the present invention can be achieved using methods standard in the art.

Another preferred delivery vehicle comprises a viral vector. A viral vector includes an isolated nucleic acid molecule useful in the present invention, in which the nucleic acid molecules are packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

Another embodiment of the present invention relates to a method to regulate biological processes, including apoptosis, by regulating the expression and/or activity of AMID. This embodiment can generally include the use (e.g., administration) of therapeutic compositions comprising one or more of AMID proteins, nucleic acid molecules comprising nucleic acid sequence encoding AMID, antibodies that specifically bind to AMID, or agonists or antagonists of AMID proteins, that are useful in a method of regulating biological processes, including apoptosis, that are mediated by or associated with the expression and biological activity of AMID.

For example, included in the present invention are methods for regulating apoptosis in a cell (increasing or decreasing), as well as for the treatment of cancer by enhancing tumor cell death (apoptosis) in a mammal. In the method of increasing apoptosis in a cell, such as a tumor cell, the method includes the step of contacting the tumor cell with at least one component selected from an AMID protein of the present invention (including fragments and homologues); nucleic acids encoding an AMID protein of the invention (including fragments and homologues); antigen-presenting cells that express a nucleic acid molecule (polynucleotide) encoding an AMID protein or homologues according to the present invention, under conditions and for a time sufficient to permit the stimulation of the apoptotic process; or any products of drug design that have the biological activity of an AMID protein according to the invention. The treatment can be combined with chemotherapy, tumor excision, radiation therapy or other cancer therapy. In the method of decreasing apoptosis in a cell, the method includes the step of contacting the cell with a compound including, but not limited to, an antibody or antigen binding fragment thereof that selectively binds to AMID, an AMID antagonist protein, a fragment of AMID that has reduced apoptosis-inducing biological activity (e.g., a competitive inhibitor); an isolated nucleic acid sequence that hybridizes under highly stringent conditions to a gene encoding AMID (e.g., anti-sense); or any product of drug design that inhibits the biological activity of AMID. In one embodiment, the expression and/or biological activity of endogenous AMID in a patient's cells is regulated by a regulatory compound administered in accordance with the present invention.

Accordingly, in one embodiment, the method of the present invention preferably regulates apoptosis in a patient, such that the patient is protected from a disease that is amenable to regulation of apoptosis, such as cancer, human immunodeficiency virus infection, autoimmune diseases, etc. As used herein, the phrase “protected from a disease” refers to reducing the symptoms of the disease; reducing the occurrence of the disease, and/or reducing the severity of the disease. Protecting a patient can refer to the ability of a therapeutic composition of the present invention, when administered to a patient, to prevent a disease from occurring and/or to cure or to treat the disease by alleviating disease symptoms, signs or causes. As such, to protect a patient from a disease includes both preventing disease occurrence (prophylactic treatment) and treating a patient that has a disease or that is experiencing initial symptoms or later stage symptoms of a disease (therapeutic treatment). The term, “disease” refers to any deviation from the normal health of a patient and includes a state when disease symptoms are present, as well as conditions in which a deviation (e.g., infection, gene mutation, genetic defect, etc.) has occurred, but symptoms are not yet manifested (e.g., a precancerous condition).

More specifically, a therapeutic composition as described herein, when administered to a patient by the method of the present invention, preferably produces a result which can include alleviation of the disease (e.g., reduction of at least one symptom or clinical manifestation of the disease), elimination of the disease, reduction of a tumor or lesion associated with the disease, elimination of a tumor or lesion associated with the disease, prevention or alleviation of a secondary disease resulting from the occurrence of a primary disease, or prevention of the disease.

According to the present invention, an effective administration protocol (i.e., administering a therapeutic composition in an effective manner) comprises suitable dose parameters and modes of administration that result in the desired effect in the patient (e.g., regulation of apoptosis in a given cell), preferably so that the patient is protected from the disease (e.g., by disease prevention or by alleviating one or more symptoms of ongoing disease). Effective dose parameters can be determined using methods standard in the art for a particular disease. Such methods include, for example, determination of survival rates, side effects (i.e., toxicity) and progression or regression of disease.

In accordance with the present invention, a suitable single dose size is a dose that results in regulation of apoptosis in a patient, or in the amelioration of at least one symptom of a condition in the patient, when administered one or more times over a suitable time period. Doses can vary depending upon the disease being treated. For example, in the treatment of cancer, a suitable single dose can be dependent upon whether the cancer being treated is a primary tumor or a metastatic form of cancer. One of skill in the art can readily determine appropriate single dose sizes for a given patient based on the size of a patient and the route of administration.

In one aspect of the invention, a suitable single dose of a therapeutic composition of the present invention is an amount that, when administered by any route of administration, regulates at least one parameter of AMID expression or biological activity in the cells of the patient as described above, as compared to a patient which has not been administered with the therapeutic composition of the present invention (i.e., a pre-determine control patient or measurement), as compared to the patient prior to administration of the composition, or as compared to a standard established for the particular disease, patient type and composition. A suitable single dose of a therapeutic composition to regulate a cancer or tumor is an amount that is sufficient to reduce, stop the growth of, and preferably eliminate, the tumor following administration of the composition into the tissue of the patient that has cancer.

As discussed above, a therapeutic composition of the present invention is administered to a patient in a manner effective to deliver the composition to a cell, a tissue, and/or systemically to the patient, whereby the desired result (e.g., increased apoptosis of tumor cells) is achieved as a result of the administration of the composition. Suitable administration protocols include any in vivo or ex vivo administration protocol. The preferred routes of administration will be apparent to those of skill in the art, depending on the type of condition to be prevented or treated; whether the composition is nucleic acid based, protein based, or cell based; and/or the target cell/tissue. For proteins or nucleic acid molecules, preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue. Routes useful for deliver to mucosal tissues include, bronchial, intradermal, intramuscular, intranasal, other inhalatory, rectal, subcutaneous, topical, transdermal, vaginal and urethral routes. Combinations of routes of delivery can be used and in some instances, may enhance the therapeutic effects of the composition.

Ex vivo administration refers to performing part of the regulatory step outside of the patient, such as administering a composition (nucleic acid or protein) of the present invention to a population of cells removed from a patient under conditions such that the composition contacts and/or enters the cell, and returning the cells to the patient. Ex vivo methods are particularly suitable when the target cell type can easily be removed from and returned to the patient.

Many of the above-described routes of administration, including intravenous, intraperitoneal, intradermal, and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art.

One method of local administration is by direct injection. Direct injection techniques are particularly useful for administering a composition to a cell or tissue that is accessible by surgery, and particularly, on or near the surface of the body. Administration of a composition locally within the area of a target cell refers to injecting the composition centimeters and preferably, millimeters from the target cell or tissue.

Various methods of administration and delivery vehicles disclosed herein have been shown to be effective for delivery of a nucleic acid molecule to a target cell, whereby the nucleic acid molecule transfected the cell and was expressed. In many studies, successful delivery and expression of a heterologous gene was achieved in preferred cell types and/or using preferred delivery vehicles and routes of administration of the present invention. All of the publications discussed below and elsewhere herein with regard to gene delivery and delivery vehicles are incorporated herein by reference in their entirety.

For example, using liposome delivery, U.S. Pat. No. 5,705,151, issued Jan. 6, 1998, to Dow et al. demonstrated the successful in vivo intravenous delivery of a nucleic acid molecule encoding a superantigen and a nucleic acid molecule encoding a cytokine in a cationic liposome delivery vehicle, whereby the encoded proteins were expressed in tissues of the animal, and particularly in pulmonary tissues. In addition, Liu et al., Nature Biotechnology 15:167, 1997, demonstrated that intravenous delivery of cholesterol-containing cationic liposomes containing genes preferentially targets pulmonary tissues and effectively mediates transfer and expression of the genes in vivo. Several publications by Dzau and collaborators demonstrate the successful in vivo delivery and expression of a gene into cells of the heart, including cardiac myocytes and fibroblasts and vascular smooth muscle cells using both naked DNA and Hemagglutinating virus of Japan-liposome delivery, administered by both incubation within the pericardium and infusion into a coronary artery (intracoronary delivery) (See, for example, Aoki et al., 1997, J. Mol. Cell, Cardiol. 29:949-959; Kaneda et al., 1997, Ann N. Y Acad. Sci. 811:299-308; and von der Leyen et al., 1995, Proc Natl Acad Sci USA 92:1137-1141).

Delivery of numerous nucleic acid sequences has been accomplished by administration of viral vectors encoding the nucleic acid sequences. Using such vectors, successful delivery and expression has been achieved using ex vivo delivery (See, of many examples, retroviral vector; Blaese et al., 1995, Science 270:475-480; Bordignon et al., 1995, Science 270:470-475), nasal administration (CFTR-adenovirus-associated vector), intracoronary administration (adenoviral vector and Hemagglutinating virus of Japan, see above), intravenous administration (adeno-associated viral vector; Koeberl et al., 1997, Proc Natl Acad Sci USA 94:1426-1431). A publication by Maurice et al. (1999, J. Clin. Invest. 104:21-29) demonstrated that an adenoviral vector encoding a P2-adrenergic receptor, administered by intracoronary delivery, resulted in diffuse multichamber myocardial expression of the gene in vivo, and subsequent significant increases in hemodynamic function and other improved physiological parameters. Levine et al. describe in vitro, ex vivo and in vivo delivery and expression of a gene to human adipocytes and rabbit adipocytes using an adenoviral vector and direct injection of the constructs into adipose tissue (Levine et al., 1998, J. Nutr. Sci. Vitaminol. 44:569-572).

In the area of neuronal gene delivery, multiple successful in vivo gene transfers have been reported. Millecamps et al. reported the targeting of adenoviral vectors to neurons using neuron restrictive enhancer elements placed upstream of the promoter for the transgene (phosphoglycerate promoter). Such vectors were administered to mice and rats intramuscularly and intracerebrally, respectively, resulting in successful neuronal-specific transfection and expression of the transgene in vivo (Millecamps et al., 1999, Nat. Biotechnol. 17:865-869). As discussed above, Bennett et al. reported the use of adeno-associated viral vector to deliver and express a gene by subretinal injection in the neural retina in vivo for greater than 1 year (Bennett, 1999, ibid.).

Gene delivery to synovial lining cells and articular joints has had similar successes. Oligino and colleagues report the use of a herpes simplex viral vector which is deficient for the immediate early genes, ICP4, 22 and 27, to deliver and express two different receptors in synovial lining cells in vivo (Oligino et al., 1999, Gene Ther. 6:1713-1720). The herpes vectors were administered by intraarticular injection. Kuboki et al. used adenoviral vector-mediated gene transfer and intraarticular injection to successfully and specifically express a gene in the temporomandibular joints of guinea pigs in vivo (Kuboki et al., 1999, Arch. Oral. Biol. 44:701-709). Apparailly and colleagues systemically administered adenoviral vectors encoding IL-10 to mice and demonstrated successful expression of the gene product and profound therapeutic effects in the treatment of experimentally induced arthritis (Apparailly et al., 1998, J. Immunol. 160:5213-5220). In another study, murine leukemia virus-based retroviral vector was used to deliver (by intraarticular injection) and express a human growth hormone gene both ex vivo and in vivo (Ghivizzani et al., 1997, Gene Ther. 4:977-982). This study showed that expression by in vivo gene transfer was at least equivalent to that of the ex vivo gene transfer. As discussed above, Sawchuk et al. has reported successful in vivo adenoviral vector delivery of a gene by intraarticular injection, and prolonged expression of the gene in the synovium by pretreatment of the joint with anti-T cell receptor monoclonal antibody (Sawchuk et al., 1996, ibid. Finally, it is noted that ex vivo gene transfer of human interleukin-1 receptor antagonist using a retrovirus has produced high level intraarticular expression and therapeutic efficacy in treatment of arthritis, and is now entering FDA approved human gene therapy trials (Evans and Robbins, 1996, Curr. Opin. Rheumatol. 8:230-234). Therefore, the state of the art in gene therapy has led the FDA to consider human gene therapy an appropriate strategy for the treatment of at least arthritis. Taken together, all of the above studies in gene therapy indicate that delivery and expression of a recombinant nucleic acid molecule according to the present invention is feasible.

Another method of delivery of recombinant molecules is in a non-targeting carrier (e.g., as “naked” DNA molecules, such as is taught, for example in Wolff et al., 1990, Science 247, 1465-1468). Such recombinant nucleic acid molecules are typically injected by direct or intramuscular administration. Recombinant nucleic acid molecules to be administered by naked DNA administration include an isolated nucleic acid molecule of the present invention, and preferably includes a recombinant molecule of the present invention that preferably is replication, or otherwise amplification, competent. A naked nucleic acid reagent of the present invention can comprise one or more nucleic acid molecules of the present invention including a dicistronic recombinant molecule. Naked nucleic acid delivery can include intramuscular, subcutaneous, intradermal, transdermal, intranasal and oral routes of administration, with direct injection into the target tissue being most preferred. A preferred single dose of a naked nucleic acid vaccine ranges from about 1 nanogram (ng) to about 100 μg, depending on the route of administration and/or method of delivery, as can be determined by those skilled in the art. Suitable delivery methods include, for example, by injection, as drops, aerosolized and/or topically. In one embodiment, pure DNA constructs cover the surface of gold particles (1 to 3 μm in diameter) and are propelled into skin cells or muscle with a “gene gun.”

In the method of the present invention, therapeutic compositions can be administered to any member of the Vertebrate class, Mammalia, including, without limitation, primates, rodents, livestock and domestic pets. Livestock include mammals to be consumed or that produce useful products (e.g., sheep for wool production). Preferred patients to protect include the mammals: humans, dogs, cats, mice, rats, sheep, cattle, horses and pigs, with humans being most preferred.

Conditions to treat using methods of the present invention include any condition, disease in which it is useful to modulate the activity of AMID. Such conditions include, but are not limited to, any condition in which regulation of apoptosis may be beneficial. Cancers to treat using the present method include, but are not limited to, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, primary hepatic cancers, lung cancers, pancreatic cancers, gastrointestinal cancers, renal cell carcinomas, hematopoietic neoplasias and metastatic cancers thereof.

Another embodiment of the invention relates to the use of any of the AMID proteins, polynucleotides or antibodies described herein to induce, prevent, or detect apoptosis in a cell.

One embodiment of the invention relates to the use of an AMID protein or nucleic acid encoding the same (including fragments and homologues), as an oxidoreductase. Such enzyme activity can be utilized in vitro or in vivo, depending on the desired application. For example, one can contact an AMID protein or biologically active fragment or homologue thereof to a suitable substrate to utilize the oxidoreductase activity of the AMID protein.

One embodiment of the present invention relates to a method to identify regulators of AMID by identifying putative regulatory compounds which increase, decrease or mimic the expression and/or biological activity of AMID proteins or nucleic acid molecules.

Such methods can be cell-free or cell-based assays. In one embodiment, a method to identify a compound that regulates AMID expression or biological activity is provided. The method includes the steps of: (a) contacting an AMID protein, a biologically active fragment thereof, a biologically active homologue thereof, or a nucleic acid molecule encoding any of such proteins with a putative regulatory compound; and (b) detecting whether the putative regulatory compound binds to and/or regulates the expression or activity of AMID (or a nucleic acid molecule encoding AMID) as compared to prior to contact with the compound. A compound that binds to or associates with the protein or nucleic acid molecule and/or that increases or decreases the expression or activity of AMID, as compared to the protein in the absence of the compound, indicates that the putative regulatory compound is a regulator of AMID. Biological activities of AMID that can be detected have been described in detail above.

In one aspect, the step of detecting comprises detecting whether AMID expression or activity is regulated by contacting cells or tissue that express AMID with the putative regulatory compound and measuring changes in AMID expression and/or biological activity in the cells or tissue. In another aspect, the step of detecting comprises detecting whether AMID-induced apoptosis is regulated in the presence of the putative regulatory compound as compared to in the absence of the putative regulatory compound.

In another related embodiment, the invention provides a method to identify an AMID homologue that regulates AMID biological activity. In this embodiment, the AMID homologue (including agonists and antagonists) is effectively the putative regulatory compound to be evaluated for activity. This method comprising detecting whether a putative AMID homologue has at least one biological activity of an AMID protein as described previously herein, or whether the putative AMID homologue can compete with native AMID in an assay (e.g., for a substrate). AMID homologues according to the invention have been described in detail above.

In one aspect of these methods, the method can include the steps of: (a) contacting an AMID protein with a putative regulatory compound; and, (b) detecting whether the AMID protein is capable of binding to the putative regulatory compound or whether AMID activity is increased or decreased in the presence of the regulatory compound as compared to in the absence of the putative regulatory compound. In these embodiments, the AMID protein can be provided as a substantially purified protein or in a cell-free extract (e.g., the membrane form separate from cell membranes), expressed by a cell, or in a cell lysate. In one embodiment, one can test a putative compound for its ability to disrupt the association of AMID with another protein (e.g., a ligand or substrate).

In yet another aspect, if a putative AMID protein homologue is to be evaluated (e.g., the homologue is the putative regulatory compound to be tested as being an agonist or antagonist of AMID), the AMID homologue may tested for one of the above-identified activities (e.g., binding or biological activities), without the need for the presence of AMID, except as a positive or negative control.

As used herein, the term “putative” refers to compounds having an unknown or previously unappreciated regulatory activity in a particular process. As such, the term “identify” is intended to include all compounds, the usefulness of which as a regulatory compound of AMID for the purposes of regulating apoptosis is determined by a method of the present invention.

The methods of the present invention include contacting components chosen from: AMID protein, an AMID homologue, and/or a putative regulatory compound with one another to detect binding of one component to another or to detect the effect of the contact on expression and/or biological activity of one or more of the components. The step of contacting can be performed by any suitable method, depending on how the components are provided. For example, cells expressing AMID can be grown in liquid culture medium or grown on solid medium in which the liquid medium or the solid medium contains the compound to be tested in the presence or absence of AMID. In addition, as described above, the liquid or solid medium contains components necessary for cell growth, such as assimilable carbon, nitrogen and micro-nutrients. Cell lysates can be combined with other cell lysates and/or the compound to be tested in any suitable medium. In another embodiment, the AMID protein, AMID homologue, and/or cell lysates containing such proteins can be immobilized on a substrate such as: artificial membranes, organic supports, biopolymer supports and inorganic supports. The protein can be immobilized on the solid support by a variety of methods including adsorption, cross-linking (including covalent bonding), and entrapment. Adsorption can be through van del Waal's forces, hydrogen bonding, ionic bonding, or hydrophobic binding. Exemplary solid supports for adsorption immobilization include polymeric adsorbents and ion-exchange resins. Solid supports can be in any suitable form, including in a bead form, plate form, or well form. The putative regulatory compound can be contacted with the immobilized protein by any suitable method, such as by flowing a liquid containing the compound over the immobilized protein.

The present methods involve contacting cells with the compound being tested for a sufficient time to allow for interaction with, activation of or inhibition of the AMID protein by the compound. The period of contact with the compound being tested can be varied depending on the result being measured, and can be determined by one of skill in the art. For example, for binding assays, a shorter time of contact with the compound being tested is typically suitable, than when activation is assessed. As used herein, the term “contact period” refers to the time period during which the proteins are in contact with the compound being tested and/or the time period during which the AMID protein and test compound are in contact (or in a condition where contact is possible) with each other. The term “incubation period” refers to the entire time during which, for example, cells are allowed to grow prior to evaluation, and can be inclusive of the contact period. Thus, the incubation period includes all of the contact period and may include a further time period during which the compound being tested is not present but during which growth is continuing (in the case of a cell based assay) prior to scoring. The incubation time for growth of cells can vary but is sufficient to allow for interaction of AMID with the test compound, followed by a response to such interaction. It will be recognized that shorter incubation times are preferable because compounds can be more rapidly screened. A preferred incubation time is between about 1 minute to about 48 hours.

The conditions under which the cell or cell lysate of the present invention is contacted with a putative regulatory compound and/or with other cells or cell lysates, such as by mixing, are any suitable culture or assay conditions and includes an effective medium in which the cell can be cultured or in which the cell lysate can be evaluated in the presence and absence of a putative regulatory compound. Similarly, the conditions under which AMID is contacted with a putative regulatory compound are any suitable assay conditions, such as by immobilization of the protein on a substrate in conditions under which the protein can contact the putative regulatory compound.

Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Acceptable protocols to contact a cell with a putative regulatory compound in an effective manner include the number of cells per container contacted, the concentration of putative regulatory compound(s) administered to a cell, the incubation time of the putative regulatory compound with the cell, and the concentration of compound administered to a cell. Determination of such protocols can be accomplished by those skilled in the art based on variables such as the size of the container, the volume of liquid in the container, the type of cell being tested and the chemical composition of the putative regulatory compound (i.e., size, charge etc.) being tested. A preferred amount of putative regulatory compound(s) comprises between about 1 nM to about 10 mM of putative regulatory compound(s) per well of a 96-well plate.

Suitable cells for use with the present invention include any cell that endogenously expresses an AMID protein or which has been transfected with and expresses recombinant AMID protein as disclosed herein (such as 293 cells, COS cells, CHO cells, fibroblasts, etc., genetically engineered to express AMID). Cells for use with the present invention include mammalian, invertebrate, plant, insect, fungal, yeast and bacterial cells. Preferred cells include mammalian cells. Preferred mammalian cells include primate, non-human primate, mouse and rat, with human cells being preferred. Preferably, the test cell (host cell) should express a functional AMID protein that is capable of inducing apoptosis in the host cell, preferably greater than 2, 5, or 10-fold induction over background.

The AMID protein can be contacted with other compounds, such as natural binding or associated proteins or substrates for AMID, either prior to, simultaneous with, or after contact of the putative regulatory compound with the cell, depending on how the assay is to be evaluated, and depending on whether activation or inhibition of AMID is to be evaluated.

As discussed above, the step of detecting whether a putative regulatory compound binds to, activates and/or inhibits AMID expression or biological activity can be performed by any suitable method, including, but not limited to measurement of AMID transcription; measurement of AMID translation; measurement of AMID ligand/substrate binding to AMID, to an AMID homologue or to a putative regulatory compound; measurement of AMID translocation within a cell; or by measuring markers of apoptosis. Such methods are known in the art, and include a variety of binding assays, western blotting, immunocytochemistry, flow cytometry, other immunological based assays, phosphorylation assays, kinase assays, immunofluorescence microscopy, RNA assays, immunoprecipitation, evaluation of cell morphology, in situ hybridization, and other biological assays. Binding assays include BlAcore machine assays, immunoassays such as enzyme linked immunoabsorbent assays (ELISA) and radioimmunoassays (RIA), or determination of binding by monitoring the change in the spectroscopic or optical properties of the proteins through fluorescence, UV absorption, circular dichrosim, or nuclear magnetic resonance (NMR).

One embodiment of the invention relates to a method to identify a compound that regulates the expression of AMID RNA, comprising: (a) contacting a putative regulatory compound with a recombinant host cell that expresses a recombinant nucleic acid molecule encoding AMID or a recombinant host cell that has been transfected with a nucleic acid sequence comprising an AMID gene regulatory region operatively linked to a reporter nucleic acid sequence; and (b) detecting whether the putative regulatory compound regulates expression of the recombinant nucleic acid molecule encoding AMID or the reporter nucleic acid sequence as compared to prior to contact with the compound. Compounds that regulate expression of the recombinant nucleic acid molecule encoding AMID or the reporter nucleic acid sequence, as compared to in the absence of the compound, indicates that the putative regulatory compound is a regulator of AMID expression. More particularly, in one embodiment, a nucleic acid sequence encoding a reporter molecule can be linked to a regulatory element of the gene encoding AMID and used in appropriate intact cells, cell extracts or lysates to identify compounds that modulate AMID expression. Appropriate cells or cell extracts can be prepared, if desired, from any cell type that normally expresses AMID, thereby ensuring that the cell extracts contain the transcription factors required for in vitro or in vivo transcription. The screen can be used to identify compounds that modulate the expression of the reporter construct. In such screens, the level of reporter gene expression is determined in the presence of the test compound and compared to the level of expression in the absence of the test compound.

According to the present invention, the method can include the step of detecting the expression of an AMID-encoding gene or expression of AMID. As used herein, the term “expression”, when used in connection with detecting the expression of a gene or nucleic acid or protein of the present invention, can refer to detecting transcription of the gene (e.g., detecting an RNA or cDNA product) and/or to detecting translation of the gene (e.g., detecting production of a protein). To detect expression of a gene refers to the act of actively determining whether a gene is expressed or not. This can include determining whether the gene expression is upregulated as compared to a control, downregulated as compared to a control, or unchanged as compared to a control. Therefore, the step of detecting expression does not require that expression of the gene actually is upregulated or downregulated, but rather, can also include detecting that the expression of the gene has not changed (i.e., detecting no expression of the gene or no change in expression of the gene). Expression of transcripts and/or proteins is measured by any of a variety of known methods in the art. For RNA expression, methods include but are not limited to: extraction of cellular mRNA and northern blotting using labeled probes that hybridize to transcripts encoding all or part of one or more of the genes of this invention; amplification of mRNA expressed from one or more of the genes of this invention using gene-specific primers and reverse transcriptase—polymerase chain reaction, followed by quantitative detection of the product by any of a variety of means; extraction of total RNA from the cells, which is then labeled and used to probe cDNAs or oligonucleotides encoding all or part of the genes of this invention, arrayed on any of a variety of surfaces. The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

In any of the above-described methods of the present invention, the AMID protein can be any AMID protein described herein, including homologues and biologically active fragments thereof.

Yet another embodiment of the invention relates to a genetically modified non-human animal comprising a genetic modification within at least one allele of the gene locus comprising the gene encoding AMID (referred to as the amid locus), wherein the genetic modification results in a reduction of AMID biological activity in the animal. In one embodiment, the animal comprises a genetic modification in both alleles of its amid locus, wherein the genetic modification results in an absence of AMID biological activity in the animal. In another embodiment, a genetically modified non-human animal is modified to overexpress a gene encoding AMID as described herein (a transgenic animal).

According to the present invention, a “genetically modified” animal has a genome which is modified (i.e., mutated or changed) from its normal (i.e., wild-type or naturally occurring) form such that the desired result is achieved (e.g., a reduction in the action of AMID or overexpression of AMID). Genetic modification of an animal is typically accomplished using molecular genetic and cellular techniques, including manipulation of embryonic cells and DNA (e.g., DNA comprising the amid locus). Such techniques are generally disclosed for mice, for example, in “Manipulating the Mouse Embryo” (Hogan et al., Cold Spring Harbor Laboratory Press, 1994, incorporated herein by reference in its entirety).

A genetically modified non-human animal can include a non-human animal in which nucleic acid molecules have been modified (i.e., mutated; e.g., by insertion, deletion, substitution, and/or inversion of nucleotides), in such a manner that such modifications provide the desired effect within the animal (i.e., reduction in AMID expression or activity or overexpression of AMID). As used herein, genetic modifications which result in a reduction in gene expression, in the function of the gene, or in the function of the gene product (i.e., the protein encoded by the gene) can be referred to as inactivation (complete or partial), deletion, interruption, blockage or down-regulation of a gene. For example, a genetic modification in a gene which results in a decrease in the function of the protein encoded by such gene, can be the result of: a partial or complete deletion of the gene or of an exon within the gene (i.e., the gene does not exist, and therefore the protein can not be produced); a mutation (e.g., a deletion, substitution, insertion and/or inversion) in the gene which results in incomplete or no translation of the protein (e.g., a mutation which causes a frame shift so that the correct protein is not expressed, a mutation in one or more exons of the gene so that the protein or at least a portion of the protein is not expressed, or a mutation in a regulatory region so that the protein is not expressed or has reduced expression); or a mutation in the gene which decreases or abolishes the natural function of the protein (e.g., a protein is expressed which has decreased or no biological activity or action).

As used herein, a non-human animal suitable for genetic modification according to the present invention is any non-human animal for which the amid locus can be manipulated, including non-human members of the Vertebrate class, Mammalia, such as non-human primates and rodents. Preferably, such a non-human animal is a rodent, and more preferably, a mouse.

Techniques for achieving targeted integration of an isolated nucleic acid molecule into a genome are well known in the art and are described, for example in “Manipulating the Mouse Embryo”, supra. For example, the isolated nucleic acid molecule can be engineered into a targeting vector which is designed to integrate into a host genome. According to the present invention, a targeting vector is defined as a nucleic acid molecule which has the following three features: (1) genomic sequence from the target locus in the host genome to stimulate homologous recombination at that locus; (2) a desired genetic modification within the genomic sequence from the target locus sufficient to obtain the desired phenotype; and (3) a selectable marker (e.g., an antibiotic resistance cassette, such as G418, neomycin, or hygromycin resistance cassettes). Such targeting vectors are well known in the art.

Following introduction of the isolated nucleic acid molecule of the targeting vector into the ES cells, ES cells which homologously integrate the isolated nucleic acid molecule are injected into mouse blastocysts and chimeric mice are produced. These mice are then bred onto the desired mouse background to detect those which transmit the mutated gene through the germ line. Heterozygous offspring of germline transmitting lines can then be mated to produce homozygous progeny.

Non-human animals which carry one or more mutated amid alleles can be identified using any suitable method for evaluating DNA. For example, genotypes can be analyzed by PCR and confirmed by Southern blot analysis as described (Sambrook et al., 1988, Molecular Cloning: A Laboratory Manual, Cold Spring Harbor Press, Cold Spring Harbor Laboratory, Cold Spring Harbor, N.Y. or Current Protocols in Molecular Biology (1989) and supplements).

Another embodiment of the invention includes a method to detect a polymorphism or a mutation in chromosome region 1 Oq21.3-q22.1, comprising incubation of a sample with an oligonucleotide comprising at least 8 nucleotides of an isolated polynucleotide from and AMID-encoding gene, homologue thereof, or a nucleic acid sequence fully complementary thereto. The effect of a polymorphism or mutation in the AMID gene sequence on the particular phenotype is determined by in vitro or in vivo assays. Generally, in vitro assays are useful in determining the direct effect of a particular polymorphism, while clinical studies will also detect a phenotype that is genetically linked to a polymorphism. Identification of mutations in AMID may be used as additional diagnostics to identify patients that are at increased risk of developing tumors due to the presence of a mutated AMID. Genotyping is performed by DNA or RNA sequence and/or hybridization analysis of any convenient sample from a patient, e.g. biopsy material, tissue sample, blood sample, scrapings from cheek, etc. A nucleic acid sample from an individual is analyzed for the presence of mutations in the AMID gene sequence, particularly those that affect the activity or expression of the AMID-encoding gene.

The following examples are provided for the purpose of illustration and are not intended to limit the scope of the present invention.

### EXAMPLES

Materials and Methods

The following materials and methods were used in Examples 1-7 below.

Reagents

The AMID EST clones (Research Genetics, Huntsville, Ala.), mouse monoclonal antibody against Flag (Sigma Company, St. Luis, Mo.) and HA (Covance, Berkeley, Calif.) epitopes, mouse monoclonal antibody against AIF (Santa Cruz Biotechnology, Santa Cruz, Calif.) and caspase-3 (Transduction Laboratories, San Diego, Calif.), Texas Red conjugated Affinipure goat anti-mouse IgG (Molecular Probes, Eugene, Oreg.), MitoTracker Green FM (Molecular Probes, Eugene, Oreg.), and caspase inhibitor VAD-fmk (Medical & Biological Laboratories, Japan) were purchased from the indicated manufactures. Human embryonic kidney 293T, monocytic U937, and T lymphoma Jurkat (Gary Johnson), wild type and p53-deficient colon cancer HCT116 cells, colon cancer DLD cells (Bert Vogelstein) were kindly provided by the indicated investigators. The B lymphoma RPM18226 cell line was purchased from ATCC (Manassas, Va.).

RNA Isolation and Northern Blot Hybridization

Human multiple tissue mRNA blots were purchased from Clontech (Palo Alto, Calif.). To detect AMID expression in cancer cell lines, total RNA was isolated by the TRIZOL (GIBCO, Rockville, Md.) method following the manufacture's protocol. The purified RNA (20 μg) was fractionated in a 1.5% agarose gel and transferred to nitrocellulose membrane as described (Sambrook (1989) Molecular Cloning: A laboratory Manual, Second Edition (Cold Spring Harbor, N.Y.: Cold Spring Harbor Laboratory Press)). The blots were hybridized with indicated probes in the Rapid Hybridization Buffer (Clontech, Palo Alto, Calif.) under high-stringency conditions.

Vector Construction and Transfection

Mammalian expression vectors for HA- or Flag-tagged AIF, AMID and their deletion mutants were constructed by PCR amplification of the corresponding cDNA fragments and subsequently cloning into CMV promoter-based vectors containing a N-terminal or C-terminal HA or Flag tag.

Transfection of 293T and COS cells was performed with standard calcium phosphate precipitation method (Sambrook, supra). Transfection of HCT116-p53(+/+) and (−/−) cells was performed with Lipofectamine (Invitrogen, Carlsbad, Calif.) by following procedures suggested by the manufacture.

Cell Fractionation and Western Blot

293 T cells (˜1×10⁷) were transfected with 20 μg of expression plasmid for c-terminal HA-tagged AMID for 16 hours. The transfected cells were harvested and centrifuged at 600 g for 10 min. The pellet was washed with 1 ml of PBS and resuspended in 300 μl of buffer A (20 mM Hepes, 10 mM KCl, 1.5 mM MgCl₂, 1 mM EDTA, 1 mM EGTA, 1 mM DTT, 0.1 mM PMSF, 250 mM sucrose, pH 7.5), homogenized by a syringe with an 18G needle for 15˜20 times. The lysate was centrifuged at 1,000 g for 10 min and the supernatant was further centrifuged at 10,000 g for 1 hr. The mitochondrion-enriched pellet was resuspended in 50 μl of Buffer A. The cytosolic supernatant (10 μl) and the mitochondrial fraction (10 μl) were fractionated on SDS-PAGE and subsequent Western blot analyses were performed as described (Hu (2000) J. Biol. Chem. 275, 10838-10844; Shu (2000) Proc. Natl. Acad. Sci. USA 97, 9156-9161).

Apoptosis Assays

β-Galactosidase co-transfection assays for determination of cell death were performed as previously described (Hsu (1996) Cell 84, 299-308; Shu (1997) Immunity 6, 751-763). Briefly, 293T cells (˜2×10⁵) were seeded on 6-well (35 mm) dishes and were transfected the following day by calcium phosphate precipitation with 0.1 μg of pCMV-β-galactosidase plasmid and the indicated testing plasmids. Within the same experiment, each transfection was performed in triplicate, and where necessary, enough amount of empty control plasmid was added to keep each transfection receiving the same amount of total DNA. Approximately 36-48 hours after transfection, the cells were stained with X-gal as previously described (Hsu, supra). The numbers of survived blue cells from five representative viewing fields were determined microscopically. Data shown are averages and standard deviations of one representative experiments in which each transfection has been performed in triplicate.

Immunofluorescent Staining

293T cells cultured on glass coverslips were sequentially plunged into methanol and acetone at −20° C., each for 10 minutes. The cells were rehydrated in PBS, blocked with 1% BSA in PBS for 15 minutes, and stained with primary antibodies (2 μg/ml) in blocking buffer for 1 hour at room temperature. The cells were rinsed with PBS, stained with Texas Red conjugated Affinipure goat anti-mouse IgG (1:200 dilution) and MitoTracker Green FM (10 μM) for 45 minutes at room temperature. The cells were then rinsed with PBS and mounted in Prolong Antifade (Molecular Probes, Eugene, Oreg.). The cells were observed with a Leica DMR/XA immunofluorescent microscope using 100× plan objective.

Electron Microscopy

Sorted GFP positive cells were fixed in 3% glutaraldehyde, stained with 1% osmium tetroxide, enrobed in seaplaque agarose, dehydrated with ethanol, and embedded in Epon/Araldite resin. Thin sections were cut, placed on butvar-coated 200 mesh copper grids, post stained with 3% aqueous uranyl acetate and Reynolds lead citrate, and observed in a Philips 400 transmission electron microscope.

### Example 1

The following example describes the identification and cloning of AMID.

To identify the novel AMID gene of the invention, the present inventors identified an EST from the GenBank database (Accession No. BG285370—a human cDNA clone derived from a prostate adenocarcinoma cell line; no protein was defined in this database submission) that appeared to the inventors to encode a protein with homology to AIF. More specifically, this search identified more than 10 EST clones that encoded a potential AIF homologous protein, which was designated as AMID (for AIF-homologous Mitochondrion-associated Inducer of Death). Sequence analysis of the longest EST clones suggested that AMID encodes a 373 aa protein (FIG. 1). The EST clone (GenBank accession number BG285370) was subsequently determined by the inventors to encode a full-length protein because there is an in-frame stop codon at the 5′ upstream of the putative ATG start codon and a poly(A) sequence at the 3′ end (data not shown). The AMID sequence was cloned into a vector as described above in the Materials and Methods. Briefly, the AMID nucleic acid sequence represented by SEQ ID NO: 1 was constructed by PCR amplification of the corresponding cDNA fragments and subsequently cloning into CMV promoter-based vectors. SEQ ID NO: 1 includes the coding region for AMID, which spans from position 215 to 1333 (plus the stop codon at positions 1334-1336) of SEQ ID NO:1, and which is represented herein by SEQ ID NO:5. SEQ ID NO:5 (and positions 215-1333 of SEQ ID NO: 1) encode the AMID protein having an amino acid sequence represented herein by SEQ ID NO:2.

Blast searches of the GeneBank databases suggested that AMID, like AIF, has significant homology with NADH-oxidoreductases/flavoproteins from bacterial to mammalian species (FIG. 1). AMID, however, does not contain a recognizable MLS that is found in AIF.

### Example 2

The following example describes the expression of AMID in human tissues.

The inventors examined expression of AMID in various human tissues by Northern blot analysis. These experiments indicated that AMID mRNA was undetectable in all tissues tested, including heart, brain, placenta, lung, liver, skeletal muscle, kidney, pancreas, spleen, thymus, prostate, testis, ovary, small intestine, colon, and peripheral blood leukocyte (data not shown). However, AMID expression could be easily detected in colon cancer cell lines DLD and HCT116, and could be weakly detected in B lymphoma cell line RPM18226 (data not shown).

To examine possible functions of AMID, mammalian expression constructs for N-terminal Flag-tagged, C-terminal HA-tagged and un-tagged AMID were produced. Briefly, 293 cells (˜2×10⁵) were transfected with 2 μg of expression plasmids for (a) AMID(1-185 of SEQ ID NO:2), (b) AMID(1-300 of SEQ ID NO:2), (c) AMID(77-373 of SEQ ID NO:2), and (d) AMID(186-373 of SEQ ID NO:2) for 16 hours, and immunofluorescent staining was performed. As expected, the N-terminal Flag-tagged AMID was expressed as a ˜43 kDa protein. Surprisingly, the C-terminal HA-tagged AMID was expressed as a ˜39 kD a protein, which was ˜4 kDa smaller than expected. Moreover, the sizes of all C-terminal tagged C-terminal deletion mutants were also ˜4 kDa smaller than expected (data not shown). It is possible that a 4 kDa N-terminal fragment in AMID is cleaved after translation. Alternatively, it is possible that AMID translation does not start from the putative start codon, but from the second or third methionine located at 37 and 41 aa downstream of the first methionine respectively (FIG. 1).

### Example 3

The following example shows that AMID is co-localized with mitochondria.

AIF has a MLS and is localized to the inter membrane space of mitochondria under physiological condition (Susin, supra; Daugas, supra). AMID, however, lacks a recognizable MLS (FIG. 1). To determine the cellular localization of AMID, immunofluorescent staining and biochemical experiments were performed. To visualize AMID localization in mammalian cells, 293 cells were transfected with expression plasmid for C-terminal HA-tagged AMID and double immunofluorescent staining with an anti-HA antibody and a fluorescent dye for mitochondria was performed. These experiments indicated that a major fraction of AMID was co-localized with mitochondria (data not shown). It seems that AMID is adhered to the outer membrane of mitochondria and forms a ring-like structure. Another fraction of AMID was localized in the cytosol. The localization of N-terminal tagged full length AMID was also examined and similar results were obtained (data not shown). To further confirm this observation, the mitochondrial and cytosolic fractions from 293T cells transfected with C-terminal HA-tagged AMID were isolated and then Western blot analysis with anti-HA antibody was performed. Results showed that AMID was detected in both the mitochondrial and the cytosolic fractions. As controls for these cell fractionation experiments, AIF was only detected in the mitochondrial fraction, while caspase-3, a cytosolic protein, was only detected in the cytosolic fraction. Taken together, these data suggest that AMID is associated with the outer membrane of mitochondria and localized in the cytosol.

### Example 4

The following example shows the induction of apoptosis by AMID.

Since AMID is an AIF homologous protein that is associated with the outer membrane of mitochondria, it was next determined whether AMID could induce apoptosis. When overexpressed, AMID-induced cell death of 293T cells in a dose dependent manner (data not shown). AMID induced cell death occurred approximately 30 hours after transfection, comparing to 14 hours for apoptosis induced by death receptors or caspase 8 (data not shown). To determine whether AMID induces apoptosis or necrosis, electron microscopy analysis was performed. Briefly, 293T cells were cotransfected with expression plasmids for AMID and green fluorescent protein (GFP). Forty hours after transfection, cells were sorted for GFP expression by flow cytometry and then analyzed by electron microscopy. Overexpression of AMID in 293T cells caused chromatin condensation and formation of apoptotic bodies (data not shown). The condensed chromatin induced by AMID was accumulated at the periphery of nucleus, a phenotype similar to that induced by AIF but distinguishable with most caspase-dependent apoptosis (Susin, supra; Daugas, supra). Interestingly, overexpression of AMID also caused loss of structurally preserved mitochondria (data not shown).

### Example 5

The following example demonstrates domain mapping of AMID-induced apoptosis.

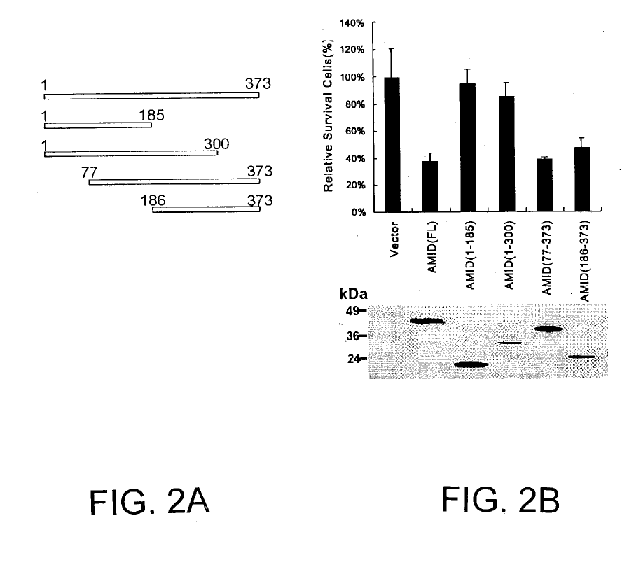

To determine the domains that are required for AMID-induced apoptosis, the inventors made a series of N- and C-terminal deletion mutants and determined their abilities to induce apoptosis. Briefly, 293T cells (˜2×10⁵) were transfected with 2 μg of expression plasmids for the indicated AMID mutants (FIG. 2B) for 16 hours and apoptosis assays were performed as described above. These experiments indicated that the C-terminal fragment (aa77-373 and aa186-373) was sufficient to induce apoptosis, while the two N-terminal fragments (aa1-185 and aa1-300) did not induce apoptosis (FIG. 2B). These data suggest that an intact flavoprotein domain spanning ˜aa80-300 is not required for AMID-induced apoptosis, which is consistent with the observation that the intact flavoprotein domain of AIF is not required for AIF-induced apoptosis (Miramar, supra). Interestingly, immunofluorescent staining indicated that aa77-373 and aa186-373 were associated with the outer membrane of mitochondria or localized in the cytosol, while aa1-185 and aa1-300 were localized in the nucleus (FIG. 2B). These data imply that the association of AMID to the outer membrane of mitochondria is critical for AMID-induced apoptosis.

### Example 6

The following example shows that AMID-induced apoptosis is independent of caspase activity and p53, and is not inhibited by Bcl2.

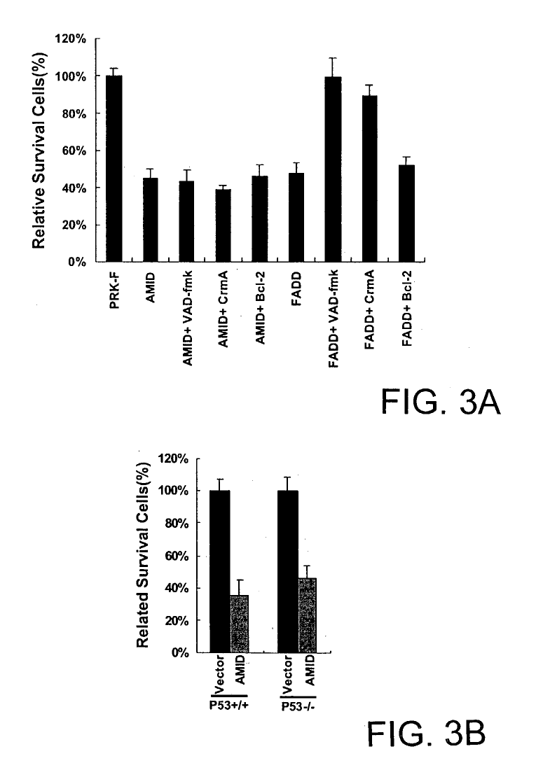

Although caspases are the central executioners of most classical apoptotic pathways, AIF-induced apoptosis is caspase-independent. The inventors investigated whether AMID-induced apoptosis is caspase-dependent. To do this, 293T cells were cotransfected with expression plasmids for AMID and crmA, a specific caspase inhibitor. Briefly, 293T cells (˜2×10⁵) were transfected with 2 μg of expression plasmid for AMID or FADD, together with 2 μg of an empty vector or an expression plasmid for crmA or Bcl2. Immediately after transfection, the cells were left untreated or treated with VAD-fmk (1 μM) for 36 hours. The cells were then stained with X-gal and survived blue cells were counted. The results indicated that crmA could not inhibit AMID-induced apoptosis (Hsu, supra) (FIG. 3A). In contrast, crmA inhibited apoptosis induced by FADD, a death domain containing protein involved in death receptor-mediated apoptosis (FIG. 3A). In addition, a pan caspase inhibitor, z-VAD.fmk, also did not inhibit AMID-induced apoptosis, but inhibited FADD-induced apoptosis (FIG. 3A). These data suggest that AMID-induced apoptosis is caspase-independent.

Bcl-2 is the prototypic member of the Bcl-2 family that can inhibit certain types of mitochondrion-mediated apoptosis. The inventors examined whether Bcl-2 could inhibit AMID-induced apoptosis. The results indicated that Bcl-2 could not inhibit AMID-induced apoptosis (FIG. 3A), pointing to the possibility that AMID functions either downstream of mitochondrion-mediated release of death-triggering molecules or in a distinct apoptotic pathway.

The inventors also examined whether AMID-induced apoptosis is p53-dependent. To do this, the effects of AMID overexpression on wild type and p53-deficient colon cancer HCT116 cells were determined. Briefly, wild type and p53-deficient HCT116 colon cells were transfected with AMID and apoptosis assays were performed as described above. As shown in FIG. 3B, overexpression of AMID induced comparable cell death in both wild type and p53-deficient HCT116 cells, suggesting that AMID-induced apoptosis can operate independent of p53.

### Example 7

The following example shows that overexpression of AIF is insufficient to induce apoptosis.

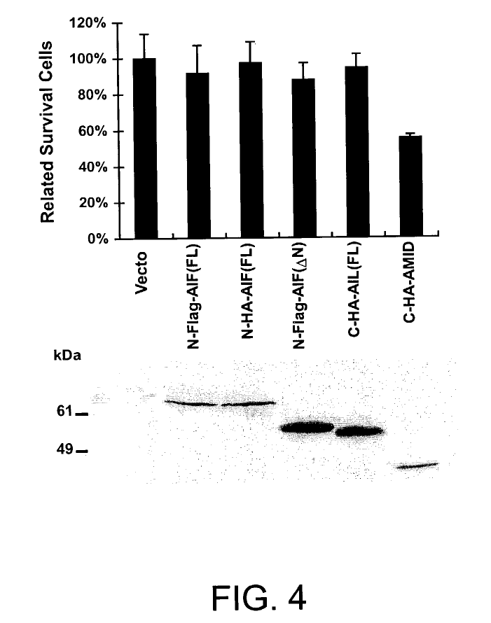

The inventors attempted to determine whether AMID is involved in AIF-induced apoptosis. However, surprisingly and in contrast with previous reports (Susin, supra; Daugas, supra; Joza, supra; Loeffler, supra), the inventors found that overexpression of AIF in 293T cells (FIG. 4) and all other cell lines tested (data not shown), including COS and embryonic fibroblasts which were used in previous reports, did not induce cell death. Briefly, 293T cells (˜2×10⁵) were transfected with 2 μg of the indicated expression plasmids for 36 hours and apoptosis assays were then performed as described above. Relative expression levels of the constructs were measured by Western blot with anti-Flag and anti-HA antibodies (FIG. 4, lower panel). Similar data were obtained by using a C-terminal tagged AIF and a AIF mutant lacking the MLS, expressed for various times ranging from 24 to 96 hours. In these experiments, AMID, which was expressed at even lower level, induced apoptosis (FIG. 4).

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. An isolated protein comprising an amino acid sequence selected from the group consisting of:
a) an amino acid sequence comprising SEQ ID NO:2;
b) a biologically active fragment of SEQ ID NO:2; and
c) an amino acid sequence that is at least about 50% identical to SEQ ID NO:2, wherein said amino acid sequence has a biological activity of SEQ ID NO:2.

2. The isolated protein of claim 1, wherein said protein comprises an amino acid sequence that is at least about 70% identical to SEQ ID NO:2, wherein said protein has a biological activity of SEQ ID NO:2.

3. The isolated protein of claim 1, wherein said protein comprises an amino acid sequence that is at least about 90% identical to SEQ ID NO:2, wherein said protein has a biological activity of SEQ ID NO:2.

4. The isolated protein of claim 1, wherein said fragment of SEQ ID NO:2 comprises an amino acid sequence spanning from a starting position of between amino acid 2 and amino acid 185 of SEQ ID NO:2, to an ending position of amino acid 373 of SEQ ID NO:2.

5. The isolated protein of claim 1, wherein said fragment of SEQ ID NO:2 is selected from the group consisting of: positions 37-373 of SEQ ID NO:2, positions 41-373 of SEQ ID NO:2, positions 77-373 of SEQ ID NO:2, and positions 186-373 of SEQ ID NO:2.

6. The isolated protein of claim 1, wherein said fragment of SEQ ID NO:2 is selected from the group consisting of: positions 77-373 of SEQ ID NO:2, and positions 186-373 of SEQ ID NO:2.

7. The isolated protein of claim 1, wherein said protein comprises an amino acid sequence that is at least 50% identical and less than 100% identical to SEQ ID NO:2, wherein said protein has a biological activity of SEQ ID NO:2.

8. The isolated protein of claim 7, wherein said protein comprises an amino acid sequence that is less than 95% identical to SEQ ID NO:2.

9. The isolated protein of claim 7, wherein said protein comprises an amino acid sequence that is less than 90% identical to SEQ ID NO:2.

10. The isolated protein of claim 1, wherein said protein comprises SEQ ID NO:2.

11. The isolated protein of claim 1, wherein said biological activity is apoptosis-inducing activity.

12. An isolated fusion protein comprising:
a) a first amino acid sequence selected from the group consisting of:
i) an amino acid sequence comprising SEQ ID NO:2;
ii) a biologically active fragment of SEQ ID NO:2; and
iii) an amino acid sequence that is at least about 50% identical to SEQ ID NO:2 and has a biological activity of SEQ ID NO:2; and
b) a second amino acid sequence that is heterologous to said first amino acid sequence, wherein said first and second are linked to form a fusion protein.

13. An isolated antibody or antigen binding fragment thereof that selectively binds to SEQ ID NO:2.

14. A diagnostic kit comprising at least one antibody or antigen binding fragment thereof of claim 13.

15. A composition comprising at least about 1 μg of an isolated protein selected from the group consisting of:
a) an amino acid sequence comprising SEQ ID NO:2;
b) a biologically active fragment of SEQ ID NO:2; and
c) an amino acid sequence that is at least about 50% identical to SEQ ID NO:2, and has a biological activity of SEQ ID NO:2.

16. An isolated nucleic acid molecule consisting essentially of a nucleic acid sequence selected from the group consisting of:
a) a nucleic acid sequence encoding SEQ ID NO:2;
b) a nucleic acid sequence encoding a biologically active fragment of SEQ ID NO:2;
c) a nucleic acid sequence encoding an amino acid sequence that is at least about 50% identical to SEQ ID NO:2 and has a biological activity of SEQ ID NO:2; and
d) a nucleic acid sequence that is fully complementary to any of the nucleic acid sequences of (a)-(c).

17. The isolated nucleic acid molecule of claim 16, wherein said nucleic acid sequence encodes an amino acid sequence that is at least about 70% identical to SEQ ID NO:2 and has a biological activity of SEQ ID NO:2.

18. The isolated nucleic acid molecule of claim 16, wherein said nucleic acid sequence encodes an amino acid sequence that is at least about 90% identical to SEQ ID NO:2 and has a biological activity of SEQ ID NO:2.

19. The isolated nucleic acid molecule of claim 16, wherein said nucleic acid sequence encodes a fragment of SEQ ID NO:2 spanning from a starting position of between amino acid 2 to amino acid 185 of SEQ ID NO:2, to an ending position of amino acid 373 of SEQ ID NO:2.

20. The isolated nucleic acid molecule of claim 16, wherein said nucleic acid sequence encodes a fragment of SEQ ID NO:2 spanning positions 77-373 of SEQ ID NO:2.

21. The isolated nucleic acid molecule of claim 16, wherein said nucleic acid sequence encodes a fragment of SEQ ID NO:2 spanning positions 186-373 of SEQ ID NO:2.

22. The isolated nucleic acid molecule of claim 16, wherein said nucleic acid sequence encodes a protein having an amino acid sequence that is at least 50% identical and less than 100% identical to SEQ ID NO:2, wherein said protein has a biological activity of SEQ ID NO:2.

23. The isolated nucleic acid molecule of claim 16, wherein said nucleic acid sequence encodes SEQ ID NO:2.

24. The isolated nucleic acid molecule of claim 16, wherein said nucleic acid molecule consists essentially of SEQ ID NO:5

25. A recombinant nucleic acid molecule comprising the isolated nucleic acid molecule of claim 16 and a heterologous vector sequence.

26. The recombinant nucleic acid molecule of claim 25, wherein said nucleic acid molecule of claim 16 is operatively linked to a transcription control sequence.

27. An isolated host cell transfected with the recombinant nucleic acid molecule of claim 25.

28. An oligonucleotide consisting essentially of at least 22 consecutive nucleotides of SEQ ID NO: 1 or SEQ ID NO:5, or the complement thereof.

29. A diagnostic kit comprising at least one oligonucleotide of claim 28.

30. A method for detecting the presence of a cancer in a patient, comprising the steps of:
a) obtaining a biological sample comprising cells from a patient;
b) detecting in said cells the expression of a protein comprising SEQ ID NO:2 or a nucleic acid molecule comprising SEQ ID NO:5; and
c) comparing the level of expression of the protein or nucleic acid molecule detected in (b) to a control expression level as an indicator of the presence of a cancer in the patient, wherein a decrease in the expression of said protein or said nucleic acid molecule as compared to the control is an indicator of a positive diagnosis of cancer in said patient.

31. The method of claim 30, wherein said step of detecting comprises contacting said biological sample with a binding agent that binds to a protein comprising SEQ ID NO:2, and detecting in the sample an amount of protein that binds to the binding agent.

32. The method of claim 31, wherein said binding agent is selected from the group consisting of: an antibody, an antigen binding fragment, and a peptide that selectively binds to SEQ ID NO:2.

33. The method of claim 31, wherein said step of contacting comprises contacting said cells in said biological sample intracellularly with said binding agent.

34. The method of claim 30, wherein said step of detecting comprises contacting nucleic acids in said biological sample with an oligonucleotide consisting essentially of at least 22 consecutive nucleotides of SEQ ID NO:5, or the complement thereof, and detecting in the sample an amount of nucleic acids that hybridize to the oligonucleotide under highly stringent conditions.

35. The method of claim 34, wherein step of contacting comprises contacting said cells in said biological sample intracellularly with said oligonucleotide.

36. The method of claim 30, wherein said step of detecting comprises detecting expression of an RNA sequence comprising SEQ ID NO:5 or a fragment thereof in said biological sample as compared to expression of said RNA sequence in said control.

37. The method of claim 30, wherein said step of detecting comprises detecting a nucleic acid sequence in a sample comprising a cDNA product of RNA comprising SEQ ID NO:5 or a fragment thereof as compared to a cDNA product of RNA in said control.

38. The method of claim 30, wherein said step of detecting comprises detecting a nucleic acid sequence in a sample comprising amplified nucleic acid products of RNA comprising SEQ ID NO:5 or a fragment thereof in said sample as compared to amplified nucleic acid products of RNA from said control.

39. A method for inducing apoptosis in a cell, comprising contacting a cell intracellularly with a compound selected from the group consisting of:
a) an isolated protein comprising an amino acid sequence selected from the group consisting of:
i) an amino acid sequence comprising SEQ ID NO:2;
ii) a biologically active fragment of SEQ ID NO:2; and
iii) an amino acid sequence that is at least about 50% identical to SEQ ID NO:2, wherein said amino acid sequence has a biological activity of SEQ ID NO:2;
b) an isolated nucleic acid molecule comprising a nucleic acid sequence encoding a protein of (a), said nucleic acid sequence being operatively linked to a transcription control sequence;
c) a product of drug discovery that has the biological activity of a protein comprising SEQ ID NO:2.

40. A method for the treatment of cancer, comprising contacting tumor cells in a patient with a compound, under conditions and for a time sufficient to permit the stimulation of the apoptotic process, said compound being selected from the group consisting of:
a) an isolated protein comprising an amino acid sequence selected from the group consisting of:
i) an amino acid sequence comprising SEQ ID NO:2;
ii) a biologically active fragment of SEQ ID NO:2; and
iii) an amino acid sequence that is at least about 50% identical to SEQ ID NO:2 and having a biological activity of SEQ ID NO:2;
b) an isolated nucleic acid molecule comprising a nucleic acid sequence encoding a protein of (a), said nucleic acid sequence being operatively linked to a transcription control sequence;
c) isolated antigen-presenting cells transfected with and expressing the isolated nucleic acid molecule of (b); and
d) a product of drug discovery that has the biological activity of a protein comprising SEQ ID NO:2.

41. The method of claim 40, wherein the treatment is combined with chemotherapy, tumor excision, radiation therapy or other cancer therapy.

42. A method for reducing apoptosis in a cell, comprising contacting a cell intracellularly with a compound selected from the group consisting of:
a) an antibody or antigen binding fragment thereof that selectively binds to and inhibits the activity of a protein comprising SEQ ID NO:2;
b) an isolated protein that is at least about 50% identical and less than about 100% identical to SEQ ID NO:2, wherein said protein is an antagonist of a protein comprising SEQ ID NO:2;
c) a fragment of SEQ ID NO:2 that has reduced apoptosis-inducing biological activity as compared to SEQ ID NO:2;
d) an isolated nucleic acid sequence comprising at least 22 consecutive nucleotides of SEQ ID NO:1 or SEQ ID NO:5 and that hybridizes under highly stringent conditions to a gene encoding SEQ ID NO:2 and inhibits the expression of a protein comprising SEQ ID NO:2; and
e) a product of drug discovery that inhibits the activity of a protein comprising SEQ ID NO:2 or the expression of a gene encoding a protein comprising SEQ ID NO:2.

43. A method to identify a regulator of apoptosis, comprising:
a) contacting an apoptosis-inducing protein or nucleic acid molecule encoding said apoptosis-inducing protein with a putative regulatory compound, wherein said apoptosis-inducing protein comprises an amino acid sequence selected from the group consisting of:
i) an amino acid sequence comprising SEQ ID NO:2;
ii) a biologically active fragment of SEQ ID NO:2; and
iii) an amino acid sequence that is at least about 50% identical to SEQ ID NO:2 and has a biological activity of SEQ ID NO:2;
b) detecting whether said putative regulatory compound increases or decreases expression or activity of said protein or said nucleic acid molecule as compared to prior to contact with said compound;
wherein compounds that increase or decrease the expression or activity of the protein or nucleic acid molecule, as compared to in the absence of said compound, indicates that said putative regulatory compound is a regulator of apoptosis.

44. The method of claim 43, wherein said apoptosis-inducing protein is expressed by a cell, and wherein said putative regulatory compound is contacted with said protein or nucleic acid molecule intracellularly.

45. The method of claim 43, wherein step of detecting comprises detecting binding or association of said putative regulatory compound with said protein or nucleic acid molecule.

46. The method of claim 43, wherein said step of detecting comprises detecting the ability of said putative regulatory compound to regulate apoptosis in a cell that expresses said protein or nucleic acid molecule.


SEQUENCE LISTING (5 sequences)

SEQ ID NO: 1 - DNA, 1524 bp, Homo sapiens
  CDS (215)..(1333)
ggccgttcggagaccagccccagcgtgccaggaccgtttccggggcctggccggggcgttgccgcggggtcggggaccagcacgagtgctgagtcacgccccgcccgggagcgcctcgggtcagtaactcgggaagacgaccaagcgggagcgggagcgggagcgggagccggagcgagagcgcgcgggcgcggccgacagtgcctgatttgagatggggtcccaggtctcggtggaatcgggagctctgcacgtggtgattgtgggtgggggctttggcgggatcgcagcagccagccagctgcaggccctgaacgtccccttcatgctggtggacatgaaggactccttccaccacaatgtggctgctctccgagcctccgtggagacagggttcgccaaaaagacattcatttcttactcggtgactttcaaggacaacttccggcaggggctagtagtggggatagacctgaagaaccagatggtgctgctgcagggtggcgaggccctgcccttctctcatcttatcctggccacgggcagcactgggcccttcccgggcaagtttaatgaggtttccagccagcaggccgctatccaggcctatgaggacatggtgaggcaggtccagcgctcacggttcatcgtggtggtgggaggaggctcggctggagtggagatggcagcagagattaaaacagaatatcctgagaaagaggtcactctcattcactcccaagtggccctggctgacaaggagctcctgccctccgtccggcaggaagtgaaggagatcctcctccggaagggcgtgcagctgctgctgagtgagcgggtgagcaatctggaggagctgcctctcaatgagtatcgagagtacatcaaagtgcagacggacaaaggcacagaggtggccaccaacctggtgattctctgcaccggcatcaagatcaacagctccgcctaccgcaaagcatttgagagcagactagccagcagtggtgctctgagagtgaacgagcacctccaggtggagggccacagcaacgtctacgccattggtgactgtgccgacgtgaggacgcccaagatggcctatcttgccggcctccacgccaacatcgccgtggccaacatcgtcaactctgtgaagcagcggcctctccaggcctacaagccgggtgcactgacgttcctcctgtccatggggagaaatgacggtgtgggccaaatcagtggcttctatgtgggccggctcatggttcggctgaccaagagccgggacctgttcgtctctacgagctggaaaaccatgaggcagtctccaccttgatggagaggccaggcgggagaactaccgcagcaggtgggcgtacggactgcttggcgcatggcacccgcctggcaagtgctagaactaatgctattcttctggaataagatgccaatgatgtggtggctagaaatgcaacttgtataaaacaaaaatgggagagagagaggtattaaacaaataccccc

SEQ ID NO: 2 - protein, 373 aa, Homo sapiens
MGSQVSVESGALHVVIVGGGFGGIAAASQLQALNVPFMLVDMKDSFHHNVAALRASVETGFAKKTFISYSVTFKDNFRQGLVVGIDLKNQMVLLQGGEALPFSHLILATGSTGPFPGKFNEVSSQQAAIQAYEDMVRQVQRSRFIVVVGGGSAGVEMAAEIKTEYPEKEVTLIHSQVALADKELLPSVRQEVKEILLRKGVQLLLSERVSNLEELPLNEYREYIKVQTDKGTEVATNLVILCTGIKINSSAYRKAFESRLASSGALRVNEHLQVEGHSNVYAIGDCADVRTPKMAYLAGLHANIAVANIVNSVKQRPLQAYKPGALTFLLSMGRNDGVGQISGFYVGRLMVRLTKSRDLFVSTSWKTMRQSPP

SEQ ID NO: 3 - protein, 425 aa, Homo sapiens
EEVPQDKAPSHVPFLLIGGGTAAFAAARSIRARDPGARVLIVSEDPELPYMRPPLSKELWFSDDPNVTKTLRFKQWNGKERSIYFQPPSFYVSAQDLPHIENGGVAVLTGKKVVQLDVRDNMVKLNDGSQITYEKCLIATGGTPRSLSAIDRAGAEVKSRTTLFRKIGDFRSLEKISREVKSITIIGGGFLGSELACALGRKARALGTEVIQLFPEKGNMGKILPEYLSNWTMEKVRREGVKVMPNAIVQSVGVSSGKLLIKLKDGRKVETDHIVAAVGLEPNVELAKTGGLEIDSDFGGFRVNAELQARSNIWVAGDAACFYDIKLGRRRVEHHDHAVVSGRLAGENMTGAAKPYWHQSMFWSDLGPDVGYEAIGLVDSSLPTVGVFAKATAQDNPKSATEQSGTGIRSESETESEASEITIPP

SEQ ID NO: 4 - protein, 377 aa, Escherichia coli
MSNGIVIIGSGFAARQLVKNIRKQDATIPLTLIAADSMDEYNKPDLSHVISQGQRADDLTRQTAGEFAEQFNLHLFPQTWVTDIDAEARVVKSQNNQWQYDKLVLATGASAFVPPVPGRELMLTLNSQQEYRACETQLRDARRVLIVGGGLIGSELAMDFCRAGKAVTLIDNAASILASLMPPEVSSRLQHRLTEMGVHLLLKSQLQGLEKTDSGIQATLDRQRNIEVDAVIAATGLRPETALARRAGLTINRGVCVDSYLQTSNTDIYALGDCAEINGQVLPFLQPIQLSAMVLAKNLLGNNTPLKLPAMLVKIKTPELPLHLAGETQRQDLRWQINTERQGMVARGVDDADQLRAFVVSEDRMKEAFGLLKTLPM

SEQ ID NO: 5 - DNA, 1119 bp, Homo sapiens
atggggtcccaggtctcggtggaatcgggagctctgcacgtggtgattgtgggtgggggctttggcgggatcgcagcagccagccagctgcaggccctgaacgtccccttcatgctggtggacatgaaggactccttccaccacaatgtggctgctctccgagcctccgtggagacagggttcgccaaaaagacattcatttcttactcggtgactttcaaggacaacttccggcaggggctagtagtggggatagacctgaagaaccagatggtgctgctgcagggtggcgaggccctgcccttctctcatcttatcctggccacgggcagcactgggcccttcccgggcaagtttaatgaggtttccagccagcaggccgctatccaggcctatgaggacatggtgaggcaggtccagcgctcacggttcatcgtggtggtgggaggaggctcggctggagtggagatggcagcagagattaaaacagaatatcctgagaaagaggtcactctcattcactcccaagtggccctggctgacaaggagctcctgccctccgtccggcaggaagtgaaggagatcctcctccggaagggcgtgcagctgctgctgagtgagcgggtgagcaatctggaggagctgcctctcaatgagtatcgagagtacatcaaagtgcagacggacaaaggcacagaggtggccaccaacctggtgattctctgcaccggcatcaagatcaacagctccgcctaccgcaaagcatttgagagcagactagccagcagtggtgctctgagagtgaacgagcacctccaggtggagggccacagcaacgtctacgccattggtgactgtgccgacgtgaggacgcccaagatggcctatcttgccggcctccacgccaacatcgccgtggccaacatcgtcaactctgtgaagcagcggcctctccaggcctacaagccgggtgcactgacgttcctcctgtccatggggagaaatgacggtgtgggccaaatcagtggcttctatgtgggccggctcatggttcggctgaccaagagccgggacctgttcgtctctacgagctggaaaaccatgaggcagtctccacct